(19) United States
(12) Patent Application Publication
(10) Pub. No.: US 2023/0287496 A1
(43) Pub. Date: Sep. 14, 2023

(54) METHODS OF ASSESSING QUALITY OF CELLS DURING A MANUFACTURING PROCESS

(71) Applicant: LifeVault Bio, Inc., Waltham, MA (US)

(72) Inventors: Jonathon B. Hamilton, Littleton, MA (US); Salvatore G. Visomi, Boston, MA (US); Usha Esseline Aaltje Beijnen, Boston, MA (US); Trevor J. Perry, Whitinsville, MA (US)

(21) Appl. No.: 18/016,669

(22) PCT Filed: Jul. 16, 2021
(86) PCT No.: PCT/US2021/041982

§ 371 Date: Jan. 17, 2023

Related U.S. Application Data
(60) Provisional application No. 63/064,794, filed on Aug. 12, 2020.
(60) Provisional application No. 63/052,803, filed on Jul. 16, 2020.

(51) Int. Cl. C12Q 1/6881; C12Q 1/6869

(52) U.S. Cl. CPC C12Q 1/6881; C12Q 1/6869; C12Q 2600/156

(57) ABSTRACT

Disclosed herein are methods for assessing the quality of cells received during various stages of a cell manufacturing process, and related methods of improving or optimizing a cell manufacturing process.

### RELATED APPLICATIONS

This application claims the benefit of U.S. Provisional Application No. 63/052,803, filed Jul. 16, 2020, and U.S. Provisional Application No. 63/064,794, filed Aug. 12, 2020, the entire teachings of which are incorporated herein by reference.

### BACKGROUND OF THE INVENTION

Cellular therapy as a modality for successful treatment of disease has existed for decades, notably in the form of blood stem cell transplant to treat a variety of hematological malignancies and immunological disorders, examples of which are described at dana-farber.org/stem-cell-transplantation-program. Patient-specific blood stem cells derived from bone marrow, mobilized peripheral blood, and cord blood have been successfully used as sources for blood stem cells to treat these diseases. With proven utility for treating various diseases, blood stem cell transplants still face many challenges in making the therapy more broadly applicable, including insufficient quantity of blood stem and progenitor cells for transplant, less intense conditioning regimens that support long-term efficacy with less toxicity, chronic GVHD, and of course relapse (Granot et al. Haematologica, 2020; 105(12):2716-2729). In an effort to address the insufficient cell supply for transplant, some companies have focused development on novel mobilizing agents and ex vivo expansion protocols.

In 2017, the first CAR-T cell therapy, KYMRIA H, was approved by the United States Food and Drug Administration for use in pediatric and young adult patients with B-cell Acute Lymphoblastic Leukemia (ALL). Since then, several additional therapies and indications have been approved utilizing this autologous cell treatment approach. The process for manufacturing these therapies is intensive and expensive, involving the isolation of donor T-cells, genetic engineering of T-cells for targeting the cancer and persistence of T-cell population, and T-cell activation and expansion (Vormittag et a., Curr Opin Biotechnol. 2018; 53:164-181). While development of allogenic/off the shelf versions of CAR-T cell therapies should reduce the cost of treatment, the manufacturing process will still rely on the core steps of isolation, engineering, activation, and expansion. Both CAR-T and blood stem cell transplants have proven successful in treating disorders originating in the blood (Goldsmith et al., Frontiers in Oncology, 2020; 10:2904), but notably are limited in treating diseases that do not originate from or exist within blood as their developmental potential is generally restricted to blood cell types.

In 1998, the first human embryonic stem cells (hESCs) were isolated in the lab of James Thomson at the University of Wisconsin-Madison (Thomson et al. Science, 1998; 282:861-872; Takahashi et al. Cell, 2007; 131:861-872). Uniquely, these cells, derived from a day 5 pre-implantation blastocyst stage during embryogenesis, were not restricted in their potential to generate the variety of cell types that exist in the human body. This state of developmental potential known as pluripotency, in combination with the hESCs unique ability to self-renew (e.g., the persistent ability to generate additional cells of identical genetic makeup and pluripotentiality), opened the door to treating diseases in all cell types and tissues in the human body.

hESCs, while harboring this unlimited therapeutic potential, also came with moral and ethical objections as their isolation resulted in the destruction of human embryos. These objections led to limited financial support, thus slowing their development towards therapeutic utility. In 2007, these objections were obviated as the labs of Shinya Yamanaka and James Thomson independently developed the ability to convert human adult/somatic skin cells to pluripotent stem cells by exogenously delivering a combination of genes associated with the embryonic cell state (Takahashi et al. Cell, 2007; 131:861-872; Yu et al. Science, 2007; 318:1917-1920). This process, known as cellular reprogramming, not only obviated the moral and ethical issues associated with using hESCs to develop new treatments, but also made it possible to create pluripotent stem cells (PSCs) from each individual. This opened the door to autologous treatment of diseases affecting all tissues of the human body, not just blood.

With this capability in hand, the field focused efforts on the development of best systems and practices to ensure compatibility with intended future uses in the development of iPSC-derived cell therapies. As such, primary somatic cell materials, cellular reprogramming systems, cell culture/expansion systems and environments, gene editing technologies, and differentiation protocols were evaluated and developed towards consistency/reproducibility, integrity, quality, and scalability in manufacturing processes. Notably, cellular reprogramming protocol development focused on the use of blood derived cell types as a primary somatic cell starting material given the ease of access in a clinical setting and the pre-existence of biobanked blood materials. Of additional benefit, blood derived cell types are better protected from environmental mutagens such as UV light, which can lead to accumulation of genetic variations/mutations in DNA sequences and chromosome structure.

Clonal Hematopoiesis of Indeterminant Potential (CHIP) is the age-related accumulation of somatic genetic variation(s) that confer(s) a competitive growth advantage to a distinct subpopulation of hematopoietic stem and progenitor cells relative to other stem and progenitor cells in the blood. Some of these somatic genetic variations have been associated with diseases, including bloodborne cancers (Genovese et al. N Engl J Med, 2014; 371(26):2477-2487) and cardiovascular disease, including aortic valve stenosis, venous thrombosis, and heart failure (Jaiswal et al. N Engl J Med, 2014; 371:2488-2498; Mas-Peiro et al. Eur Heart J, 2019; 41:933-939; Sano et al. Jacc Basic Transl Sci, 2019; 4:684-697; Bazeley et al. Curr Hear Fail Reports, 2020; 17:271-276).

Given that the vast majority of cells and cell and gene therapy products are developed from cell types derived from blood (e.g., HSCs, T-cells and primary cells for iPSC generation) and/or are delivered to the blood compartment for treatment of a disease, and that genetic variations associated with CHIP uniquely accumulate and expand in the DNA of blood cells, including, as further described herein, during the manufacture of cell and gene therapy products, there is a definitive need to identify and eliminate the accumulation of these genetic variations in both the cellular materials entering or resulting from the manufacturing process, as well as the process itself.

### SUMMARY OF THE INVENTION

There is a definitive need to identify and remove genetic variations from cell therapy manufacturing processes and the materials that result therefrom, thereby inherently reducing the risk and unknown impact of disease associated genetic variations on the health of an individual receiving the therapy.

Described herein are methods of assessing quality of cells during a manufacturing process, e.g., at multiple stages of a manufacturing process. The methods may generally include receiving a sample of cells at one or more time points during a manufacturing process; sequencing at least part of the genome of one or more cells received at the one or more time points; and identifying in the received cells a defect in one or more genes, for example, one or more genes selected from the group consisting of DNMT3A, TET2, ASXL1, PPM1D, JAK2, TP53, SRSF2, KRAS and SF3B1.

Also described herein are methods of evaluating quality of cells. The methods include receiving a sample of pluripotent or somatic cells prior to a manufacturing process; sequencing at least part of the genome of the pluripotent or somatic cells; and identifying in the pluripotent or somatic cells a defect in one or more genes, for example, one or more genes selected from the group consisting of DNMT3A, TET2, ASXL1, PPM1D, JAK2, TP53, SRSF2, KRAS and SF3B1.

Further described herein are methods of evaluating quality of cells, where the methods include receiving a sample of starter cells prior to a manufacturing process, wherein the starter cells are, comprise or consist of HSCs or T cells; sequencing at least part of the genome of the starter cells; and identifying in the starter cells a defect in one or more genes, for example, one or more genes selected from the group consisting of DNMT3A, TET2, ASXL1, PPM1D, JAK2, TP53, SRSF2, KRAS and SF3B1.

Also described herein are methods of evaluating quality of manufactured cells, e.g., cells manufactured from a population of pluripotent cells, somatic cells, hematopoietic stem cells (HSCs), or T cells. The methods include receiving a sample of manufactured cells obtained upon completion of a manufacturing process; sequencing at least part of the genome of the manufactured cells; and identifying in the manufactured cells a defect in one or more genes, for example, one or more genes selected from the group consisting of DNMT3A, TET2, ASXL1, PPM1D, JAK2, TP53, SRSF2, KRAS and SF3B1.

In some embodiments, the sample of cells is received at one or more time points during the manufacturing process selected from the group consisting of: receipt of starter cells, completion of one or more stages of manipulation of the cells (e.g., one or more of culture and expansion, genetic manipulation, differentiation, heterogeneity/subtyping, harvest, cryopreservation, thawing, isolation, enrichment, single cell cloning, and purification), and receipt of manufactured cells prior to use. In one embodiment, cellular reprogramming of the cells comprises converting an isolated somatic primary cell to an induced pluripotent stem cell. In one embodiment, the manipulation of the cells comprises manipulating a T cell to a CAR T cell. The CAR T cell may be engineered to target an antigen of interest on a cancer cell or on a tumor cell.

In some embodiments, the genetic manipulation comprises manipulating cells using one or more of CRISPR, TALEN, Zn-Finger, and vector delivery systems. The gene editing system may be delivered to a cell via a vector delivery system (such as a RNA, DNA, or viral vector delivery system). In some embodiments, the genetic manipulation is selected from the group consisting of correcting one or more genetic defects, reducing expression of one or more genes, and increasing expression of one or more genes. In one embodiment, the genetic manipulation comprises inactivating or knocking out TET2.

In some embodiments, differentiation comprises converting a starter cell (e.g., an HSC) into a therapeutic cell type. In some embodiments, the starter cell comprises a pluripotent cell and/or the therapeutic cell type is selected from the group consisting of beta cells, cardiomyocytes, satellite cells, retinal cells, NK cells, and neural cells. In some embodiments, differentiation comprises converting a pluripotent cell into a therapeutic cell type (e.g., beta cells, motor neuron cells, cardiomyocytes, satellite cells, NK cells, neural cells, etc.).

In some embodiments, one or more cells in the manufacturing process are manufactured from a population of starter cells. The starter cells may be stem cells. In some embodiments, the starter cells are pluripotent cells (e.g., induced pluripotent stem cells (iPSCs) and/or embryonic stem cells (ESCs)) or somatic cells. In other embodiments, the starter cells are hematopoietic stem cells (HSCs) or T cells. In some embodiments, the starter cells are obtained from a blood sample. For example, the population of starter cells is obtained from a subject, such as a subject in need thereof or a donor subject (e.g., a healthy donor).

In some embodiments, the defect is a sequence-based mutation, e.g., a mis-sense mutation, silent mutation, frame-shift mutation, nonsense mutation, insertion mutation, deletion mutation, or splice-site disruption. In some embodiments, the defect is a somatic sequence-based mutation or a germline sequence-based mutation. In one embodiment, the defect is in DNMT3A in exons 7 to 23. In one embodiment, the defect is a mis-sense mutation in DNMT3A selected from the group consisting of G543C, S714C, F732C, Y735C, R736C, R749C, F751C, W753C, and L889C. In one embodiment, the defect is a V617F mutation in JAK2. In one embodiment, the defect is a disruptive mutation in TET2. In one embodiment, the defect is a disruptive mutation in PPM1D. In one embodiment, the defect is a mis-sense mutation in TP53 selected from the group consisting of R175H, G245S, R248W, R273G, P151S, R181H, H193R, M237I, G245C, R248Q, R267W, and R273L.

In some embodiments, the one or more genes are associated with tumorigenesis. The one or more genes may be selected from the group consisting of TP53, KRAS, ASXL1, JAK2, SFSR2, and SFSB1, and more specifically are selected from the group consisting of TP53 and KRAS. In some embodiments, the one or more genes are associated with cancer. The one or more genes may be selected from the group consisting of DNMT3A, TET2, ASXL1, PPM1D, JAK2, SF3B1, SRSF2, and TP53, and more specifically are selected from the group consisting of DNMT3A, TET2, and ASXL1. In some embodiments, the one or more genes are associated with blood cancer, and may be selected from the group consisting of TET2 and DNMT3A.

In some embodiments, the methods described herein include identifying in the received cells a defect (e.g., a sequence-based mutation) in one or more genes selected from the group consisting of PCM1, HIF1A, and APC. In some embodiments, the methods described herein further comprise identifying in the received cells a defect in one or more genes selected from the group consisting of TERT and CHEK2. In some embodiments, the methods described herein further comprise identifying in the received cells a defect in one or more genes selected from the group consisting of CBL, KMT2C, ATM, CHEK2, KDR, MGA, DNMT3B, ARID2, SH2B3, MPL, RAD21, SRSF2, and CCND2. In some embodiments, the methods described herein further comprise identifying in the received cells a defect in one or more genes selected from the group consisting of HPRT, JAK1, JAK3, SLAMF6, IRF1, PLRG1, STAT3, and Notch1.

In some embodiments, the methods described herein include identifying in the received cells a structure-based mutation in one or more genes selected from the group consisting of DNMT3A, TET2, ASXL1, PPM1D, JAK2, TP53, SRSF2, KRAS and SF3B1. In some embodiments, the structure-based mutation is a duplication, deletion, copy number variation, inversion, or translocation. In some embodiments, the structure-based mutation occurs on one or more chromosomes selected from the group consisting of Ch2, Ch4, Ch9, Ch12, Ch17, and Ch20, and more specifically on one or more chromosomes selected from the group consisting of Ch2p23, Ch4q24, Ch20q11, Ch17q23, Ch9p24, Ch17p23, Ch17q25, Ch2q33, and Ch12p12.

In one embodiment, a structure-based mutation of DNMT3A occurs on chromosome 2p23. In one embodiment, a structure-based mutation of TET2 occurs on chromosome 4q24. In one embodiment, a structure-based mutation of ASXL1 occurs on chromosome 20q11. In one embodiment, a structure-based mutation of PPMD1 occurs on chromosome 17q23. In one embodiment, a structure-based mutation of JAK2 occurs on chromosome 9p24. In one embodiment, a structure-based mutation of TP53 occurs on chromosome 17p13. In one embodiment, a structure-based mutation of SRSF2 occurs on chromosome 17q25. In one embodiment, a structure-based mutation of SF3B1 occurs on chromosome 2q33.

In some embodiments, the methods described herein further include identifying in the received cells a structure-based mutation occurring on one or more chromosomes selected from the group consisting of Ch1, Ch12, Ch17q, Ch20q11, and X-chromosome. In some embodiments, the methods described herein further include identifying in the received cells a structure-based mutation occurring on one or more chromosomes selected from the group consisting of Ch3, Ch4, Ch5, Ch7, Ch8, Ch9, Ch11, Ch12, Ch13, Ch14, and Ch18.

In one embodiment, the sample of cells comprises iPSCs derived from a blood sample of a subject in need of treatment. In an alternative embodiment, the sample of cells comprises iPSCs derived from a blood sample of a donor subject (e.g., a healthy donor subject). In some embodiments, the sample of cells comprises hematopoietic stem cells derived from a blood sample of a subject in need of treatment. In one embodiment, the sample of cells comprises hematopoietic stem cells derived from a blood sample of a donor subject. In one embodiment, the sample of cells comprises T cells derived from a blood sample of a subject in need of treatment or from a donor subject. In some embodiments, the sample of cells is a sample of manufactured cells.

In some embodiments, the methods described herein further include identifying one or more time points during the manufacturing process wherein a defect in the one or more genes is identified. In some embodiments, the methods described herein further include isolating a subpopulation of received cells that exhibit no identified defects in the one or more genes. In some embodiments, the methods described herein further include subjecting the isolated subpopulation of received cells to the cell therapy manufacturing process.

In some embodiments, the methods described herein further include isolating a subpopulation of received cells that exhibit a defect in the one or more genes. In some embodiments, the methods described herein further include correcting the defect in the one or more genes. In some embodiments, the methods described herein further include subjecting the corrected isolated subpopulation of received cells to the cell therapy manufacturing process.

In some embodiments, the methods described herein further include isolating a subpopulation of the manufactured cells that exhibit no identified defects in the one or more genes. In some embodiments, the methods described herein further include administering to a subject the isolated manufactured cells that exhibit no identified defects in the one or more genes. In some embodiments, the isolated manufactured cells are administered to the subject to treat a disease or disorder. In some embodiments, the disease or disorder is a blood, immune, metabolic, neurologic, or cardiovascular disorder. In some embodiments, the disease or disorder is cancer. In some embodiments, the disease or disorder is selected from the group consisting of acute myeloid leukemia, acute lymphoblastic leukemia, myelodysplastic syndrome, myeloproliferative neoplasm, germ cell tumor, neuroblastoma, Ewing sarcoma, and medulloblastoma. In some embodiments, the disease or disorder is a solid tumor (e.g., a non-malignant or malignant tumor).

In other embodiments, the methods described herein further include isolating a subpopulation of manufactured cells that exhibit a defect in the one or more genes. In some embodiments, the methods described herein further include correcting the defect in the one or more genes. In some embodiments, the methods described herein further include administering to a subject the corrected isolated manufactured cells.

Also described herein are methods of maintaining quality of cells during a manufacturing process. The methods may include sequencing at least part of a genome of one or more iPSC donor cells from a subject; identifying in the donor cells a defect in one or more genes selected from the group consisting of DNMT3A, TET2, ASXL1, PPM1D, JAK2, TP53, SRSF2, KRAS and SF3B1; isolating the donor cells that exhibit no identified defects in the one or more genes; subjecting the isolated donor cells to a cell therapy manufacturing process to produce one or more manufactured cells; sequencing at least part of the genome of the one or more manufactured cells; identifying in the manufactured cells a defect in one or more genes selected from the group consisting of DNMT3A, TET2, ASXL1, PPM1D, JAK2, TP53, SRSF2, KRAS and SF3B1; and isolating the manufactured cells that exhibit no identified defects in the one or more genes.

Further disclosed herein are methods of maintaining quality of cells during a manufacturing process. The methods include sequencing at least part of a genome of one or more HSC or T cell donor cells from a subject; identifying in the donor cells a defect in one or more genes selected from the group consisting of DNMT3A, TET2, ASXL1, PPM1D, JAK2, TP53, SRSF2, KRAS and SF3B1; isolating the donor cells that exhibit no identified defects in the one or more genes; subjecting the isolated donor cells to a cell therapy manufacturing process to produce one or more manufactured cells; sequencing at least part of the genome of the one or more manufactured cells; identifying in the manufactured cells a defect in one or more genes selected from the group consisting of DNMT3A, TET2, ASXL1, PPM1D, JAK2, TP53, SRSF2, KRAS and SF3B1; and isolating the manufactured cells that exhibit no identified defects in the one or more genes.

The methods may further include a step of sequencing at least part of the genome of the isolated donor cells during one or more stages of the cell therapy manufacturing process; identifying in the cells in the manufacturing process a defect in one or more genes selected from the group consisting of DNMT3A, TET2, ASXL1, PPM1D, JAK2, TP53, SRSF2, KRAS and SF3B1; and isolating the cells in the manufacturing process that exhibit no identified defects in the one or more genes.

In some embodiments, the isolated cells are subjected to one or more additional stages of the cell therapy manufacturing process. In some embodiments, the methods described herein further include a step of administering to the subject the isolated manufactured cells that exhibit no identified defects in the one or more genes. In some embodiments, the isolated manufactured cells are administered to the subject to treat a disease or disorder. In some embodiments, the disease or disorder is a blood, immune, metabolic, neurologic, or cardiovascular disorder. In some embodiments, the disease or disorder is a cancer. In some embodiments, the disease or disorder is selected from the group consisting of acute myeloid leukemia, acute lymphoblastic leukemia, myelodysplastic syndrome, myeloproliferative neoplasm, germ cell tumor, neuroblastoma, Ewing sarcoma, and medulloblastoma. In some embodiments, the disease or disorder is a solid tumor, e.g., a non-malignant tumor or a malignant tumor.

The above discussed, and many other features and attendant advantages of the present inventions will become better understood by reference to the following detailed description of the invention.

### BRIEF DESCRIPTION OF THE FIGURES

The patent or application file contains at least one drawing executed in color. Copies of this patent or patent application publication with color drawings will be provided by the Office upon request and payment of the necessary fee.

FIG. 1 provides a flowchart of a cell manufacturing workflow for iPSC-based cell therapies.

FIG. 2 provides a flowchart of a cell manufacturing workflow for CAR-T cell therapies.

### DETAILED DESCRIPTION OF THE INVENTION

Cell therapy requires the administration of cells to a patient for the purposes of treating a disease or disorder, such as cancer. It is beneficial to assess cells prior to administration to minimize administering cells containing mutations or defects. In addition, the manipulation of cells during a manufacturing process to produce therapeutic cells can include many steps, each of which can result in accumulation of DNA damage and/or mutation, including sequence and/or structural damage or mutations. It would be beneficial to identify any DNA damage in cells prior to manipulating the cells or administering the cells to a patient. Further, it would be beneficial to identify at what time points during the manufacturing process that the cells accumulate DNA damage and, if possible, isolate and remove or repair the damaged cells, such that as manipulation progresses the resultant cells do not demonstrate the accumulated damage.

Described herein are methods for assessing the quality of cells, e.g., pluripotent cells, hematopoietic stem cells, or T cells. For example, the quality of cells may be assessed upon removal from a subject, prior to administration of a subject, or during a manufacturing process. Also described herein are methods of maintaining the quality of cells during a manufacturing process. In certain embodiments, disclosed herein are methods of evaluating quality of cells, e.g., cells at the beginning of the manufacturing process, cells removed at one or more time points during the manufacturing process, and/or cells resulting from the manufacturing process. In some embodiments, a sample of cells is received and at least part of the genome of one or more cells is sequenced. In some embodiments, defects in one or more genes selected from the group of genes consisting of DNMT3A, TET2, ASXL1, PPM1D, JAK2, TP53, SRSF2, KRAS and SF3B1 are identified in the received cells.

In some aspects, defects are somatic sequence mutations and/or germline sequence mutations. In some embodiments, somatic sequence mutations in one or more genes are identified in a sample of cells. In some embodiments, germline sequence mutations are identified in a sample of cells. In some embodiments, the sample of cells is further assessed for structure-based mutations (e.g., somatic structural chromosomal mutations), such as by microarray analysis. In some aspects, somatic structural chromosomal mutations are identified in a sample of cells.

Sequencing of DNA can be performed on tissues or cells. Sequencing of specific cell types can identify mutations in specific cell types that provide specific predictive value. Some cell types may provide a greater predictive value than other cell types. Sequencing can also be conducted in single cells, using appropriate single-cell sequencing strategies. Single-cell analyses can be used to identify high-risk combinations of mutations co-occurring in the same cells. Co-occurrence signifies that the mutations are occurring in the same cell clone and carry a greater risk, and therefore have a greater predictive value, than occurrence of the same mutations in different individual cells.

In some embodiments, at least part of the genome of one or more cells in a sample is sequenced. In some embodiments the part of the genome that is sequenced is limited to specific genes, the whole exome, or parts of an exome. For example, in certain aspects, the sequencing may be whole exome sequencing (WES). Sequencing can be carried out according to any suitable technique. Many proprietary sequencing systems are available commercially and can be used in the context of the present invention, such as for example from Illumina, USA. Exemplary single-cell sequencing methods may include those described, for example, by Eberwine et al., Nature Methods 11, 25-27 (2014) doi:10.1038/nmeth.2769 Published online 30 Dec. 2013; and especially single cell sequencing in microfluidic droplets (Nature 510, 363-369 (2014) doi:10.1038/nature13437), the entire contents of which are incorporated herein by reference.

Sequencing may be performed of specific genes only, specific parts of the genome, or the whole genome. In some aspects, specific parts of a gene can be sequenced, for example, in DNMT3A exons 7 to 23 can be sequenced. Where a part of a genome is sequenced, that part can be the exome. The exome is the part of the genome formed by exons, and thus an exon sequencing method sequences the expressed sequences in the genome. There are 180,000 exons in the human genome, which constitute about 1% of the genome, or approximately 30 million base pairs. Exome sequencing requires enrichment of sequencing targets for exome sequences, and several techniques can be used, including PCR, molecular inversion probes, hybrid capture of targets, and solution capture of targets. Sequencing of targets can be conducted by any suitable technique.

Methods of identifying structural mutations (e.g., somatic structural chromosomal mutations) and germline sequence mutations in cell samples are known to those of skill in the art. Exemplary methods are described in WO 2019/079493 and US 2017/0321284, the contents of which are incorporated herein by reference.

In some embodiments, a defect or mutation (e.g., a defect in one or more genes selected from the group consisting of DNMT3A, TET2, ASXL1, PPM1D, JAK2, TP53, SRSF2, KRAS and SF3B1) is identified in a sample of cells (e.g., a sample of cells provided from a donor or provided from one or more discreet time points of a cell therapy manufacturing process). In some aspects, a defect or mutation is identified in one or more genes selected from the group consisting of DNMT3A, TET2, ASXL1, PPM1D, JAK2, TP53, SRSF2, and SF3B1.

In some aspects, a defect or mutation is identified in one or more genes associated with tumorigenesis (e.g., one or more genes selected from the group of genes consisting of TP53, KRAS, ASXL1, JAK2, SFSR2, and/or SFSB1). In one aspect, a defect or mutation is identified in TP53 and/or KRAS. In some aspects, a defect or mutation is identified in one or more genes associated with blood cancer (e.g., TET2 and/or DNMT3A).

In some aspects, certain genes may be designated as high impact genes (e.g., DNMT3A, TET2 and/or ASXL1) or low impact genes (e.g., PPM1D, JAK2, SF3B1, SRSF2 and/or TP53). High impact genes are those that will have a more significant impact when exhibiting a defect than low impact genes. In one aspect, a defect or mutation is identified in DNMT3A, TET2, and/or ASXL1.

In certain embodiments, a defect in TP53 is identified (e.g., in a sample of cells). In certain embodiments, a defect in KRAS is identified (e.g., in a sample of cells). In certain embodiments, a defect in TET2 is identified (e.g., in a sample of cells). In certain embodiments, a defect in DNMT3A is identified (e.g., in a sample of cells). In certain embodiments, a defect in ASXL1 is identified (e.g., in a sample of cells).

DNMT3A is DNA cytosine-5-1-methyltransferase 3 alpha and is encoded on chromosome 2 (HGMC 2978). ASXL1 is additional sex combs like transcriptional regulator 1 and is encoded on chromosome 20 (HGNC 18318). TET2 is tet methylcytosine dioxygenase 2 and is encoded on chromosome 4 (HGNC 25941). PPM1D is protein phosphatase, Mg2+/Mn2+ dependent, 1D and is encoded on chromosome 17 (HGNC 9277). JAK2 is janus kinase 2 and is encoded on chromosome 9 (HGNC 6192). TP53 is tumor protein p53 and is encoded on chromosome 17 (HGNC 11998). SRSF2 is serine and arginine rich splicing factor 2 and is encoded on chromosome 17 (HGNC 10783). KRAS is KRAS proto-oncogene and is encoded on chromosome 12 (HGNC 6407). SF3B1 is splicing factor 3b subunit 1 and is encoded on chromosome 2 (HGNC 10768).

In some embodiments, a defect or mutation is further identified in one or more genes selected from the group consisting of PCM1, HIF1A, and APC. In some embodiments, a defect or mutation is further identified in one or more genes selected from the group consisting of TERT and CHEK2.

In some embodiments, a defect or mutation is further identified in one or more cancer driver genes. In one embodiment, a defect or mutation is further identified in one or more genes selected from the group consisting of CBL, KMT2C, ATM, CHEK2, KDR, MGA, DNMT3B, ARID2, SH2B3, MPL, RAD21, SRSF2, and CCND2. In some embodiments, a defect or mutation is further identified in one or more genes associated with malignancy during T cell clonal expansion. In one embodiment, a defect or mutation is further identified in one or more genes selected from the group consisting of HPRT, JAK1, JAK3, SLAMF6, IRF1, PLRG1, STAT3, and Notch1.

PCM1 is pericentriolar material 1 and is encoded on chromosome 8 (HGNC 8727). HIF1A is hypoxia inducible factor 1 subunit alpha and is encoded on chromosome 14 (HGNC 4910). APC is APC regulator of WNT signaling pathway and is encoded on chromosome 5 (HGNC 583). TERT is telomerase reverse transcriptase and is encoded on chromosome 5 (HGNC 11730). CHEK2 is checkpoint kinase 2 and is encoded on chromosome 22 (HGNC 16627). CBL is Cbl proto-oncogene and is encoded on chromosome 11 (HGNC 1541). KMT2C is lysine methyltransferase 2C and is encoded on chromosome 7 (HGNC 13726). ATM is ATM serine/threonine kinase and is encoded on chromosome 11 (HGNC 795). KDR is kinase insert domain receptor and is encoded on chromosome 4 (HGNC 6307). MGA is MAX dimerization protein MGA and is encoded on chromosome 15 (HGNC 14010). DNMT3B is DNA methyltransferase 3 beta and is encoded on chromosome 20 (HGNC 2979). ARID2 is AT-rich interaction domain 2 and is encoded on chromosome 12 (HGNC 18037). SH2B3 is SH2B adaptor protein 3 and is encoded on chromosome 12 (HGNC 29605). MPL is MPL proto-oncogene, thrombopoietin receptor and is encoded on chromosome 1 (HGNC 7217). RAD21 is RAD21 cohesin complex component and is encoded on chromosome 8 (HGNC 9811). CCND2 is cyclin D2 and is encoded on chromosome 12 (HGNC 1583). HPRT is hypoxanthine phosphoribosyltransferase and is encoded on chromosome X (HGNC 5157). JAK1 is Janus kinase 1 and is encoded on chromosome 1 (HGNC 6190). JAK3 is Janus kinase 3 and is encoded on chromosome 19 (HGNC 6193). SLAMF6 is SLAM family member 6 and is encoded on chromosome 1 (HGNC 21392). IRF1 is interferon regulatory factor 1 and is encoded on chromosome 5 (HGNC 6116). PLRG1 is pleiotropic regulator 1 and is encoded on chromosome 4 (HGNC 9089). STAT3 is signal transducer and activator of transcription 3 and is encoded on chromosome 17 (HGNC 11364). Notch1 is notch receptor 1 and is encoded on chromosome 9 (HGNC 7881).

Mutations in genes can be disruptive (e.g., they have an observed or predicted effect on protein function) or non-disruptive. A non-disruptive mutation is typically a mis-sense mutation, in which a codon is altered such that it codes for a different amino acid, but the encoded protein is still expressed. In some embodiments, somatic mutations may be mis-sense mutations or disruptive mutations (e.g., frame-shift, nonsense, or splice-site disruptions).

Putative somatic mutations include but are not limited to those alleles that comprise at least one of non-silent/disruptive nucleotide changes, indels, mis-sense mutations, frameshifts, stop mutations (addition or deletion), read-through mutations, splice mutations; and a confirmed change not due to a sequencing error or artifact of the testing system.

In some embodiments, mutations in DNMT3A are predominantly mis-sense mutations. In some aspects, mutations (e.g., mis-sense mutations) in DNMT3A are localized in exons 7 to 23. In some aspects, mutations in DNMT3A are enriched for cysteine-forming mutations. A common base-pair change in somatic variants is a cytosine-to-thymine transition. In some embodiments, a mutation in DNMT3A is a mis-sense mutation selected from the group consisting of G543C, S714C, F732C, Y735C, R736C, R749C, F751C, W753C, and L889C. In some embodiments, mutations in TET2 and/or PPM1D are disruptive mutations. In some embodiments, a mutation in JAK2 is a V617F mutation. In some embodiments, a mutation in TP53 is a mis-sense mutation. In some aspects, a mutation in TP53 is a mis-sense mutation selected from the group consisting of R175H, G245S, R248W, R273G, P151S, R181H, H193R, M237I, G245C, R248Q, R267W, and R273L. Additional non-limiting examples of mutations found in DNMT3A, TET2, ASXL1, PPM1D, JAK2, TP53, SRSF2, KRAS and SF3B1 are described in: Merkle, et al., Nature, 2017, 545(7653):229-233; Avior et al., Cell Stem Cell, 2019, 25(4):456-461; Gore et al., Nature, 2011, 471(7336):63-67; Assou et al., Stem Cell Reports, 2020, 14(1):1-8; Laurent et al., Cell Stem Cell, 2011, 8(1):106-118; Mandai et al., N Engl J Med, 2017, 376(11):1038-1046; Martincorena, et al., Science, 2015, 349(6255):1483-1489 (correction published Science, 2016, 351(6277)); US 2017/0321284; and WO 2019/079493, all incorporated herein by reference. Non-limiting examples of mutations in HPRT, JAK1, JAK3, SLAMF6, IRF1, PLRG1, STAT3, and Notch1 are described in Finette et al., Leukemia, 2001, 15(12):1898-1905; Bellanger et al., Leukemia, 2014, 28(2):417-419; Savola et al., Nat Commun., 2017, 8:15869; and Blackburn, et al., Leukemia, 2012, 26:2069-2078, all incorporated herein by reference.

Structure-based mutations (e.g., structural chromosomal mutations) may include, for example, duplications, deletions, copy number variations, inversions, and/or translocations. In some embodiments, one or more structure-based mutations are identified in one or more cells in a cell sample (e.g., one or more cells provided or sampled from a cell therapy manufacturing process). In some embodiments, structure-based mutations occur on one or more chromosomes selected from the group consisting of Ch2, Ch4, Ch9, Ch12, Ch17, and Ch20. In certain embodiments, structure-based mutations occur on one or more chromosomes selected from the group consisting of Ch2p23, Ch4q24, Ch20q11, Ch17q23, Ch9p24, Ch17p23, Ch17q25, Ch2q33, and Ch12p12. In some embodiments, a structure-based mutation is further identified on one or more chromosomes selected from the group consisting of Ch1, Ch4, Ch5, Ch7, Ch8, Ch9, Ch11, Ch12, Ch15, Ch17, Ch19, Ch20, Ch22, and ChX. In some embodiments, a structure-based mutation is further identified on one or more chromosomes selected from the group consisting of Ch1, Ch12, Ch17q, Ch20q11, and ChX. In some embodiments, a structure-based mutation is further identified on one or more chromosomes selected from the group consisting of Ch3, Ch4, Ch5, Ch7, Ch8, Ch9, Ch11, Ch12, Ch13, Ch14, and Ch18 (Loh et al., “Monogenic and polygenic inheritance become instruments for clonal selection,” Nature, 2020, available at doi.org/10.1038/s41586-020-2430-6 incorporated herein by reference). In one embodiment, a structure-based mutation is further identified on one or more chromosomes selected from the group consisting of 9p, 12, 13q, and 14q.

In one embodiment, a structure-based mutation of DNMT3A occurs on chromosome 2p23. In one embodiment, a structure-based mutation of TET2 occurs on chromosome 4q24. In one embodiment, a structure-based mutation of ASXL1 occurs on chromosome 20q11. In one embodiment, a structure-based mutation of PPMD1 occurs on chromosome 17q23. In one embodiment, a structure-based mutation of JAK2 occurs on chromosome 9p24. In one embodiment, a structure-based mutation of TP53 occurs on chromosome 17p13. In one embodiment, a structure-based mutation of SRSF2 occurs on chromosome 17q25. In one embodiment, a structure-based mutation of SF3B1 occurs on chromosome 2q33.

Non-limiting examples of structure-based mutations are described in Assou et al., Stem Cell Reports, 2020, 14(1):1-8; Laurent et al., Cell Stem Cell, 2011, 8(1):106-118; Lefort et al. Nat Biotechnol, 2008, 26:1364-1366; International Stem Cell Initiative et al., Nat Biotechnol, 2011, 29:1132-1144; Varela et al., J Clin Invest, 2012, 122:569-574; Avery et al., Stem Cell Reports, 2013, 1:379-386; and Nguyen et al., Mol Hum Reprod, 2014, 20, 168-177, all incorporated herein by reference.

In some embodiments, a sample of cells comprises one or more cells for assessment, e.g., by sequencing and/or microarray analysis. The sample of cells may be a sample of one or more somatic cells or one or more hematopoietic stem cells. In some embodiments, the sample of cells comprise one or more pluripotent cells, e.g., pluripotent stem cells. In some embodiments, the sample of cells comprises one or more induced pluripotent stem cells (iPSCs) or embryonic stem cells (ESCs). In some embodiments, the sample of cells comprise one or more hematopoietic cells, e.g., hematopoietic stem cells, or T cells. In some embodiments, the sample of cells comprises one or more hematopoietic stem cells (HSCs). In some embodiments, the sample of cells comprises one or more T cells, e.g., CAR T cells.

In some embodiments, the sample of cells comprises a population of primary cells, such as epithelial cells, fibroblasts, keratinocytes, melanocytes, endothelial cells, muscle cells, hematopoietic stem cells, and mesenchymal stem cells. In some embodiments, the sample of cells is obtained from a tissue sample (e.g., from a subject, such as a human). In some embodiments, the sample of cells is obtained from a blood sample (e.g., from a subject, such as a human). A blood sample may comprise any type of blood obtained from a subject, such as, from the bone marrow, peripheral blood, or umbilical cord blood. In certain embodiments, the blood sample comprises cord blood. In certain embodiments, the sample of cells is obtained from a blood sample of a subject in need of treatment. For example, the sample of cells may comprise pluripotent cells, HSCs, or T cells (e.g., iPSCs, HSCs, or T cells derived from a blood sample of a human subject in need of treatment). In other embodiments, the sample of cells is obtained from a blood sample of a donor subject (e.g., a subject who is donating cells for delivery to a subject in need of treatment). For example, the sample of cells may comprise pluripotent cells (e.g., iPSCs, derived from a blood sample of a donor subject), HSCs (e.g., HSCs derived from a blood sample of a donor subject), or T cells (e.g., T cells derived from a blood sample of a donor subject).

In some embodiments, the sample of cells comprises one or more cells that are obtained after manipulating a pluripotent cell. In some embodiments, the sample of cells comprises one or more cells that are obtained after manipulated an HSC or T cell. For example, the sample of cells may include one or more cells obtained at the beginning of a manufacturing process, during a manufacturing process, and/or upon completion of a manufacturing process. In some embodiments, the sample of cells is sampled or provided from one or more discreet time points during a cell manufacturing process (e.g., at the beginning and/or conclusion of a cell therapy manufacturing process, or at one or more intermediate time points during such manufacturing process).

In some embodiments, a sample of cells is received at one or more time points or phases during a manufacturing or manipulation process. In some embodiments, the sample of cells is received prior to the manufacturing process beginning, at one or more time points during the manufacturing process, and/or upon completion of the manufacturing process (e.g., prior to use). In one aspect, a sample of cells is received prior to the manufacturing process (e.g., are starter or primary cells). For example, the starter cells may be somatic. In some aspects, the starter cells are a primary cell, e.g., a fibroblast. In other aspects, the starter cells are pluripotent cells (e.g., iPSCs or ESCs). In other aspects, the starter cells may be hematopoietic stem cells (HSCs) or T cells.

In certain embodiments, cells from the sample that are identified as harboring one or more mutations or defects (e.g., one or more putative sequence-based or structure-based mutations) are isolated and are not subject to further manufacturing or manipulation. In certain embodiments, cells from the sample that are identified as harboring one or more mutations or defects (e.g., one or more putative sequence-based or structure-based mutations) are isolated and are not administered to a subject in need thereof. Similarly, in certain aspects, cells from the sample that are identified as not harboring one or more mutations or defects (e.g., one or more sequence-based or structure-based mutations) are subjected to further manufacturing or manipulation. In certain aspects, cells from the sample that are identified as not harboring one or more mutations or defects (e.g., one or more sequence-based or structure-based mutations) are administered to a subject in need thereof. For example, primary or starter cells that are identified as not harboring one or more mutations or defects may be identified, isolated and subjected to a cell therapy manufacturing process, or in certain embodiments subjected to further processing in connection with cell therapy manufacturing. In some aspects, defects identified in the one or more genes of the cells (e.g., CAR T cells) from the sample may be markers or indicators of a disease, e.g., cytokine release syndrome.

In some embodiments, a manufacturing process comprises one or more stages of manipulation of a population of cells. In some aspects, the one or more stages include cellular reprogramming, culture and expansion, genetic manipulation, differentiation, heterogeneity/subtyping, harvest, cryopreservation, thawing, isolation, enrichment, single cell cloning, and purification. See Magnusson et al., PLoS One, 2013, 8(1):e53912; Choi et al. Biotechnol J., 2015, 10(10):1529-1545; Kumar et al., Trends Mol Med., 2017, 23(9):799-819; Naldini et al., EMBO Mol Med., 2019, 11(3):e9958; Eaves et al., Blood, 2015, 125(17):2605-2613; Almeida et al., Pathobiology, 2014, 81(5-6):261-275; Crisan et al., Development, 2016, 143(24):4571-4581; Park et al., Blood Res., 2015, 50(4):194-203, each of which is incorporated herein by reference.

Cellular reprogramming may include converting a somatic cell (e.g., an isolated somatic primary cell) to a pluripotent stem cell (e.g., an iPSC). In some aspects, iPSCs are derived from the blood of a subject. iPSCs may be derived from endothelial progenitor cells (EPCs), B-cells, T-cells, or generally CD34+ cells.

The culture and expansion of cells, e.g., pluripotent stem cells (PSCs), hematopoietic stem cells (HSCs) or T cells, may generate large quantities of cells for cell banking, for entry into genetic manipulation, for entry into differentiation, or for administration to a subject in need thereof. For example, a large quantity of HSCs may be generated for hematopoietic stem cell transplant.

Genetic manipulation may occur using one or more gene editing systems, including clustered regularly interspaced short palindromic repeats (CRISPR), transcription activator-like effector nucleases (TALENs), and zinc finger nucleases (ZFN). A gene editing system may be delivered to a cell using one or more vector delivery systems, such as a RNA, DNA, or viral vector delivery system. Non-limiting examples of viral vector delivery systems including retrovirus, lentivirus, adenovirus, adeno-associated virus and herpes simplex virus. In some aspects, the genetic manipulation of one or more cells includes correcting one or more genetic defects (e.g., by repairing a mutation in a somatic or germline sequence), reducing expression of one or more genes (e.g., by inactivating or deleting one or more genes), or increasing expression of one or more genes (e.g., by activating or inserting one or more genes).

Differentiation may include converting a pluripotent cell, e.g., an iPSC or ESC, into a therapeutic cell. Non-limiting examples of differentiated therapeutic cells include beta cells, cardiomyocytes, satellite cells, retinal cells, NK cells, and neural cells. A therapeutic cell type for differentiation may be selected based on the desired end use of the cells. In other aspects, differentiation may include converting a hematopoietic stem cell or T cell into a therapeutic cell. In some aspects, a T cell is manipulated to form a CAR T cell.

In some embodiment, a population of cells are manipulated or manufactured from a starting population of cells. A sample of cells may be obtained or received from the starting population of cells, the genome of one or more cells may be sequenced, and a defect may be identified in one or more genes. Additionally, or alternatively, a sample of cells may be received at one or more time points during the manipulation of the cells, the genome of one or more cells may be sequenced, and a defect may be identified in one or more genes. Lastly, a sample of cells may be obtained from the final population of manipulated cells, the genome of one or more cells may be sequenced, and a defect may be identified in one or more genes. In certain aspects, a sample of cells may be further assessed to identify a structure-based defect in one or more genes.

In some aspects, upon identification of a defect (e.g., sequence-based or structure-based) in one or more genes in the one or more cells, a subpopulation of the cells exhibiting the defect may be isolated. In certain aspects, a sequence-based defect in the subpopulation of cells is corrected, for example by gene editing, such as by using CRISPR, TALEN, or ZFN. In some aspects, upon identification of a defect in one or more genes in the one or more cells, a subpopulation of the cells not exhibiting the defect may be isolated. The corrected subpopulation of cells and/or the subpopulation of cells not exhibiting the defect may be further manipulated during the manufacturing process. Alternatively, the corrected subpopulation of cells and/or the subpopulation of cells not exhibiting the defect, e.g., the defect-free cells, may be administered to a subject in need thereof.

In one embodiment, a population of somatic cells are reprogrammed to a population of iPSCs. A sample of cells may be obtained or received from the initial population of somatic cells, the genome of one or more cells may be sequenced, and a defect may be identified in one or more genes. Additionally, or alternatively, a sample of cells may be received at one or more time points during the reprogramming of the somatic cells to iPSCs, the genome of one or more cells may be sequenced, and a defect may be identified in one or more genes. Lastly, a sample of cells may be received from the derived iPSCs, the genome of one or more cells may be sequenced, and a defect may be identified in one or more genes. A subpopulation of cells may be isolated at any stage. The subpopulation of cells may comprise one or more cells exhibiting a defect in one or more genes. In some aspects, the defect in the isolated subpopulation of cells is corrected. Alternatively, the subpopulation of cells may comprise one or more cells not exhibiting a defect in one or more genes. In some aspects, an isolated subpopulation of cells obtained during the reprogramming process not exhibiting a defect (e.g., the defect has been corrected or there was no defect) is further subjected to the reprogramming process. In some aspects, an isolated population of cells obtained from the derived iPSCs not exhibiting a defect is further submitted to a manufacturing process, such as differentiation of the iPSC to a therapeutic cell type.

In another embodiment, a population of T cells are manipulated to form a population of CAR T cells. In some aspects, the CAR T cells are first generation, second generation, third generation, or fourth generation CAR T cells. A sample of cells may be obtained or received from the initial population of T cells, the genome of one or more cells may be sequenced, and a defect may be identified in one or more genes. Additionally, or alternatively, a sample of cells may be received at one or more time points during the manipulation of the T cells to CAR T cells, the genome of one or more cells may be sequenced, and a defect may be identified in one or more genes. Lastly, a sample of cells may be received from the CAR T cells, the genome of one or more cells may be sequenced, and a defect may be identified in one or more genes. A subpopulation of cells may be isolated at any stage. In some embodiments, a CAR T cell is manipulated to modify TET2, thereby improving the immunotherapeutic benefit of the CAR T cells. For example, a CAR T cell manipulated to disrupt TET2, such as knocking down TET2, demonstrates improved therapeutic efficacy. TET2 may be inactivated by any methods known to those of skill in the art (e.g., CRISPR, TALEN, ZFN). In some embodiments, the epigenome of CAR T cells is modified to improve efficacy and persistence of the CAR T cells. The subpopulation of cells may comprise one or more cells exhibiting a defect in one or more genes. In some aspects, the defect in the isolated subpopulation of cells is corrected. Alternatively, the subpopulation of cells may comprise one or more cells not exhibiting a defect in one or more genes. In some aspects, an isolated subpopulation of cells obtained during the manipulation process not exhibiting a defect (e.g., the defect has been corrected or there was no defect) is further subjected to the manipulation process. In some aspects, an isolated population of cells obtained from the CAR T cells not exhibiting a defect is further submitted to a manufacturing process, such as culturing and expanding the cells.

In some embodiments, manufactured or manipulated cells are administered to a subject in need thereof. In some embodiments, the manufactured cells are therapeutic cells and are administered to a subject in need thereof for treating one or more diseases. In some embodiments, manufactured cells are administered to a subject in need thereof for treating diabetes, a neurodegenerative disease (e.g., Parkinson's disease), macular degeneration, spinal injury, muscle damage, or cardiac repair. In some embodiments, the manufactured cells are therapeutic cells, e.g., CAR T cells, and are administered to a subject in need thereof for treating one or more diseases. In some embodiments, manufactured cells are administered to a subject in need thereof for treating cancer, e.g., a hematologic malignancy. In some embodiments, manufactured cells are administered to a subject in need thereof for treating a tumor, e.g., a malignant or non-malignant tumor. In some embodiments, a population of cells, e.g., HSCs, obtained from a donor are administered to a subject in need thereof. In some embodiments, a population of HSCs, e.g., HSCs that do not express a putative defect in one or more genes are administered to a subject in need thereof. The HSCs may be administered via a hematopoietic stem cell transplant (HSCT). In some aspects, the subject in need thereof has undergone chemotherapy or radiotherapy prior to administration. In some embodiments, the subject is suffering from cancer. In some aspects, the subject is suffering from multiple myeloma, lymphoma, acute myeloid leukemia, acute lymphoblastic leukemia, myelodysplastic syndrome, and myeloproliferative neoplasm. In some aspects, the subject is suffering from a solid tumor, such as a germ cell tumor, neuroblastoma, Ewing sarcoma, or medulloblastoma. In one embodiment, the population of HSCs is obtained from the subject in need thereof (i.e., autologous). In alternative embodiments, the population of HSCs is obtained from a donor subject (i.e., allogenic).

As used herein, the terms “treat, “treatment,” “treated,” “treating,” etc. refer to providing medical or surgical attention, care, or management to an individual. For example, the individual is usually ill or injured, or at increased risk of becoming ill relative to an average member of the population and in need of such attention, care, or management. Treating can refer to prolonging survival as compared to expected survival if not receiving treatment. Thus, one of skill in the art realizes that a treatment may improve the disease but may not be a complete cure for the disease. Alternatively, treatment is “effective” if the progression of a disease is reduced or halted. “Treatment” can also mean prolonging survival as compared to expected survival if not receiving treatment.

In some aspects, the methods described herein are used to assess the quality of cells (e.g., therapeutic cells produced during a manufacturing process, such as a cell therapy manufacturing process). In certain embodiments, a therapeutic cell is manufactured for a known expected use. In some embodiments, defects identified in the one or more genes of the received cells have little to no impact on the risk of accumulating disease-causing mutations specific to the expected use of the manufactured therapeutic cell. In other embodiments, defects identified in the one or more genes of the received cells have an increased impact on the risk of accumulating disease-causing mutations specific to the expected use of the manufactured therapeutic cell. In some aspects, defects identified in the one or more genes of the received cells have an increased impact on the risk of accumulating disease-causing mutations associated with cardiovascular disease, blood cancer, or decreased mortality.

In some aspects, defects identified in the one or more genes of the received cells have little to no impact on the risk of accumulating disease-causing mutations specific to the expected use of the manufactured therapeutic cell, but may have an increased risk of being tumorigenic in solid tissue. For example, defects in TP53 in therapeutic cells generated for the treatment of kidney or pancreatic function will have minimal increased risk for accumulating disease specific mutations to the target tissue, but such defects are highly tumorigenic in the kidney and pancreas. In other embodiments, defects identified in the one or more genes of the received cells have an increased impact on the risk of accumulating disease-causing mutations specific to the expected use of the manufactured therapeutic cell but exhibit a low risk for tumorigenesis in solid tissue. For example, defects identified in one or more of ASXL1, JAK2, KRAS, SFSR2, and SF3B1 in iPSC-derived blood stem cells for the treatment of a blood or immune disorder may be associated with an increased risk for blood cancer and/or cardiovascular disease but exhibit a low risk for tumorigenesis in solid tissues.

In certain embodiments, the subject is a mammal, e.g., a primate, e.g., a human. The terms, “patient” and “subject” are used interchangeably herein. Preferably, the subject is a mammal. The mammal can be a human, non-human primate, mouse, rat, dog, cat, horse, or cow, but are not limited to these examples. In certain embodiments, the subject is a human.

The methods disclosed herein may be further used to improve a cell manufacturing or manipulation process (e.g., a cell therapy manufacturing process). In certain aspects, the methods disclosed herein provide a means of monitoring a population of cells as they progress through a cell manufacturing process to identify steps in such a process that cause or otherwise contribute to the accumulation of genetic defects in such cells. By identifying specific steps or processes during cell manufacturing that cause or otherwise contribute to the accumulation of genetic defects in the subject cells, the inventions disclosed herein may be used to intervene in and optimize such a manufacturing process. For example, if a population of cells is found to accumulate one or more genetic defects during a step of the manufacturing process (e.g., cells accumulate a genetic defect during one or more of cell harvest, cryopreservation, thawing, isolation, enrichment, single cell cloning and/or purification), the manufacturing process may be modified to eliminate such step or to replace such step with an alternative step that does not cause the cells to accumulate the genetic defect. As such, the methods disclosed herein provide a valuable opportunity to optimize cell manufacturing and thereby reduce the costs associated with cell manufacturing.

It is to be understood that the invention is not limited in its application to the details set forth in the description or as exemplified. The invention encompasses other embodiments and is capable of being practiced or carried out in various ways. Also, it is to be understood that the phraseology and terminology employed herein is for the purpose of description and should not be regarded as limiting.

While certain compounds, compositions and methods of the present invention have been described with specificity in accordance with certain embodiments, the following examples serve only to illustrate the methods and compositions of the invention and are not intended to limit the same.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or the entire group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, (e.g., in Markush group or similar format) it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. The publications and other reference materials referenced herein to describe the background of the invention and to provide additional detail regarding its practice are hereby incorporated by reference.

### EXEMPLIFICATION

### Example 1A: Generating iPSCs from Somatic Cells (Cellular Reprogramming)

iPSC clones are obtained as a result of a cellular reprogramming process from somatic cells. A primary screen of the iPSC clones is performed to identify sequence-based or structure-based defects in DNMT3A, TET2, ASXL1, PPM1D, JAK2, TP53, SRSF2, KRAS and/or SF3B1. Upon completion of the screen, an iPSC clone is selected that does not exhibit any sequence-based or structure-based defects, such as somatic mutations. The iPSC clones selected may then be further manipulated, such as through genetic editing or differentiation processes. However, if upon completion of the primary screen, no iPSC clones are identified that do not exhibit any sequence-based or structure-based defects, i.e., they exhibit at least one defect, then a secondary screen may be performed. To perform the secondary screen, one or more iPSC clones are selected, single cell isolated in culture wells, and expanded to generate complete clonal colonies. The complete clonal colonies are then screened for sequence-based or structure-based defects, such as somatic mutations. Upon completion of the secondary screen, the best clone(s) is/are identified that do not include any sequence-based or structure-based defects is selected for further manipulation, such as differentiation or genetic editing processes, and following which they may be subject to further screening to confirm the absence of such defects.

### Example 1B: Assessing Manufactured Cells and Use of Those Cells (Cell Quality Control Release)

Cells produced by a manufacturing process may be screened to identify sequence-based or structure-based defects in DNMT3A, TET2, ASXL1, PPM1D, JAK2, TP53, SRSF2, KRAS and/or SF3B1. Upon completion of the screen, a manufactured cell is selected that does not exhibit any sequence-based or structure-based defects, such as somatic mutations. The manufactured cells selected may then be administered to a patient in need thereof. However, if upon completion of the screen, no manufactured cells are identified that do not exhibit any sequence-based or structure-based defects, i.e., they exhibit at least one defect, then those cells would not be delivered to a patient. Depending on the type of manufactured cell and on the disease being treated, the defects in the manufactured cell clones may be assessed to determine if they pass a pre-determined threshold for failure. Considerations for establishing the failure threshold level include severity of disease, risk to patient, and the availability alternative forms of treatment. For example, defects in certain genes may have tumorigenic effect, or defects in other genes may exhibit an increased impact on the risk of accumulating disease-causing mutations.

### Example 1C: Genetic Engineering of Cells May Lead to Defects

A somatic cell may be genetically engineered prior to use, such as prior to reprogramming to a PSC. After completion of the genetic engineering, one or more clones may be selected, singled cell isolated in culture wells, expanded to generate complete clonal colonies, and screened for sequence-based or structure-based defects in DNMT3A, TET2, ASXL1, PPM1D, JAK2, TP53, SRSF2, KRAS and/or SF3B1. Upon completion of the screen, the best clone(s) that does not exhibit any sequence-based or structure-based defects is selected for further manipulation, such as cellular reprogramming. If sequence-based or structure-based defects are identified, then later rounds of genetic editing of somatic cells may be modified so as to limit any defects and to optimize the process. For example, different genetic editing technology may be used, cloning culture and/or environment may be modified, etc.

Additionally, a PSC cell may be genetically engineered, e.g., to correct a disease-causing genetic defect or to add a functional copy of a gene, prior to use, such as prior to use in a manufacturing process (e.g., differentiation). In other instances, a PSC cell obtained from a subject in need of treatment may be genetically engineered to correct a disease-causing mutation. After completion of the genetic engineering one or more clones may be selected, singled cell isolated in culture wells, expanded to generate complete clonal colonies, and screened for sequence-based or structure-based defects in DNMT3A, TET2, ASXL1, PPM1D, JAK2, TP53, SRSF2, KRAS and/or SF3B1. Upon completion of the screen, the best clone(s) that does not exhibit any sequence-based or structure-based defects is selected for further manipulation, such as differentiation into a therapeutic cell. If sequence-based or structure-based defects are identified, then later rounds of genetic editing of PSCs may be modified so as to limit any defects and to optimize the process. For example, different genetic editing technology may be used, cloning culture and/or environment may be modified, etc.

Further, a differentiated cell may be genetically engineered prior to use, such as prior to administration to a subject in need thereof. After completion of the genetic engineering one or more cells may be selected, singled cell isolated in culture wells, expanded to generate complete clonal colonies, and screened for sequence-based or structure-based defects in DNMT3A, TET2, ASXL1, PPM1D, JAK2, TP53, SRSF2, KRAS and/or SF3B1. Upon completion of the screen, the best cell(s) that does not exhibit any sequence-based or structure-based defects is selected for further manipulation or for administration to a patient in need thereof. If sequence-based or structure-based defects are identified, then later rounds of genetic editing of differentiated cells may be modified so as to limit any defects and to optimize the process. For example, different genetic editing technology may be used, cloning culture and/or environment may be modified, etc.

### Example 1D: Individual Stages of Manufacturing Process May Lead to Defects

After reprogramming a somatic cell to an iPSC there is typically a need to increase the number of iPSCs available for further manipulation, such as for differentiation into a therapeutic cell. To increase the number of iPSCs the cell culture may be scaled-up. During or upon completion of the culture scale-up one or more cells may be selected, singled cell isolated in culture wells, expanded to generate complete clonal colonies, and screened for sequence-based or structure-based defects in DNMT3A, TET2, ASXL1, PPM1D, JAK2, TP53, SRSF2, KRAS and/or SF3B1. Upon completion of the screen, the best cell(s) that does not exhibit any sequence-based or structure-based defects is selected for further manipulation, such as differentiation into a therapeutic cell. If sequence-based or structure-based defects are identified, then the cell culture conditions may be modified so as to limit any defects and to optimize the process.

After scale-up of the iPSCs the cells may be harvested for further manipulation. Upon completion of the harvest of the iPSCs one or more cells may be selected, singled cell isolated in culture wells, expanded to generate complete clonal colonies, and screened for sequence-based or structure-based defects in DNMT3A, TET2, ASXL1, PPM1D, JAK2, TP53, SRSF2, KRAS and/or SF3B1. Upon completion of the screen, the best cell(s) that does not exhibit any sequence-based or structure-based defects is selected for further manipulation, such as differentiation into a therapeutic cell. If sequence-based or structure-based defects are identified, then the harvest conditions may be modified so as to limit any defects and to optimize the process.

During additional stages of the manipulation of iPSC cells and/or the manufacture of therapeutic cells, one or more cells may be selected, singled cell isolated in culture wells, expanded to generate complete clonal colonies, and screened for sequence-based or structure-based defects in DNMT3A, TET2, ASXL1, PPM1D, JAK2, TP53, SRSF2, KRAS and/or SF3B1. Upon completion of the screen, the best cell(s) that does not exhibit any sequence-based or structure-based defects is selected for further manipulation, such as differentiation into a therapeutic cell. If sequence-based or structure-based defects are identified, then the conditions may be modified so as to limit any defects and to optimize the process.

### Example 2A: Manufacturing CAR T Cells and Assessing for Defects

CAR T cells are manufactured from blood cells obtained from a donor. The blood cells (i.e., blood mononuclear cells) are collected from the donor and are processed to isolate T cells. The cells are then screened to identify any sequence-based or structure-based defects in DNMT3A, TET2, ASXL1, PPM1D, JAK2, TP53, SRSF2, KRAS and/or SF3B1. Upon completion of the screen, T cells are selected that do not exhibit any sequence-based or structure-based defects, such as somatic mutations. The selected T cells are then activated and expanded using methods known to those of skill in the art, including those described in Wang, et al., “Clinical manufacturing of CAR T cells: foundation of a promising therapy,” Molecular Therapy—Concolytics (2016) 3, 16015 (incorporated herein by reference in its entirety). After the T cells are activated and expanded they can be screened again for any sequence-based or structure-based defects in DNMT3A, TET2, ASXL1, PPM1D, JAK2, TP53, SRSF2, KRAS and/or SF3B1. T cells that do not exhibit any sequence-based or structure-based defects are selected for genetic modification. The selected T cells are genetically modified using a viral or non-viral gene transfer system to express a chimeric antigen receptor. Viral systems may include the use of γ-retroviral vectors, lentiviral vectors, or the transposon/transposase system. Non-viral systems may include mRNA transfer-mediated gene expression. The manufactured CAR T cells are screened for any sequence-based or structure-based defects in DNMT3A, TET2, ASXL1, PPM1D, JAK2, TP53, SRSF2, KRAS and/or SF3B1.

In some instances, the manufactured CAR T cells are further genetically modified to inactivate or knock out specific genes (e.g., TET2) or to correct disease-causing genetic defects. Specific genetic modifications may be selected based on the desired end use of the manufactured CAR T cells, e.g., treating a blood cancer, a tumor, or some other disease or disorder, generating allogenic CAR T cells, etc. Each instance of genetic modification may introduce additional defects into one or more genes, and so the cells may be screened one or more times to identify any sequence-based or structure-based defects in DNMT3A, TET2, ASXL1, PPM1D, JAK2, TP53, SRSF2, KRAS and/or SF3B1.

### Example 2B: Assessing Manufactured Cells and Use of Those Cells (Cell Quality Control Release)

Cells produced by a manufacturing process may be screened to identify sequence-based or structure-based defects in DNMT3A, TET2, ASXL1, PPM1D, JAK2, TP53, SRSF2, KRAS and/or SF3B1. Upon completion of the screen, if none of the cells exhibit any sequence-based or structure-based defects, such as somatic mutations, then the manufactured cells may be administered to a patient in need thereof. However, if upon completion of the screen, manufactured cells are identified that exhibit any sequence-based or structure-based defects, i.e., they exhibit at least one defect, then those cells would not be delivered to a patient. In some instances, the cells may be remanufactured from an earlier time point during the manufacturing process prior to any defects being identified. Depending on the type of manufactured cell and on the disease being treated, the defects in the manufactured cell clones may be assessed to determine if they pass a pre-determined threshold for failure. Considerations for establishing the failure threshold level include severity of disease, risk to patient, and the availability alternative forms of treatment. For example, defects in certain genes may have tumorigenic effect, or defects in other genes may exhibit an increased impact on the risk of accumulating disease-causing mutations.

### Example 2C: Autologous Therapy (HSC)

Hematopoietic stem cells (HSCs) may be removed from a patient in need thereof, e.g., a patient in need of a hematopoietic stem cell transplant (HSCT). The received cells are screened to identify sequence-based or structure-based defects in DNMT3A, TET2, ASXL1, PPM1D, JAK2, TP53, SRSF2, KRAS and/or SF3B1. Upon completion of the screen, HSCs are selected that do not exhibit any sequence-based or structure-based defects, such as somatic mutations. The HSCs selected may then be expanded and, if still defect free after a further screen, can be administered to a patient in need thereof.

However, if upon completion of the screen, no HSCs are identified that do not exhibit any sequence-based or structure-based defects, i.e., they exhibit at least one defect, then those cells may be further assessed. For example, the HSCs may be genetically modified to address the defect in the one or more genes. Alternatively, the HSCs containing the defect(s) may be assessed to determine if the benefit of administering a patient's own cells outweighs the risks of the identified defect. For example, if the defect exhibits an increased impact on the risk of accumulating disease-causing mutations, then the HSCs may not be administered back to the donor, but instead HSCs from an allogenic donor may be obtained.

### Example 3

Cellular therapy as a modality for successful treatment of disease has existed for decades, notably in the form of blood stem cell transplant to treat a variety of hematological malignancies and immunological disorders, examples of which are described at dana-farber.org/stem-cell-transplantation-program. Patient-specific blood stem cells derived from bone marrow, mobilized peripheral blood, and cord blood have been successfully used as sources for blood stem cells to treat these diseases. With proven utility for treating various diseases, blood stem cell transplants still face many challenges in making the therapy more broadly applicable, including insufficient quantity of blood stem and progenitor cells for transplant, less intense conditioning regimens that support long-term efficacy with less toxicity, chronic GVHD, and of course relapse (1). In an effort to address the insufficient cell supply for transplant, some companies have focused development on novel mobilizing agents and ex vivo expansion protocols.

For example, a focus of efforts in the field has been on the development of best systems and practices to ensure compatibility with intended future uses in development of iPSC-derived cell therapies. As such, primary somatic cell materials, cellular reprogramming systems, cell culture/expansion systems and environments, gene correction technologies and differentiation protocols have been evaluated and developed towards consistency/reproducibility, integrity, quality and scalability in manufacturing processes. Notably, cellular reprogramming protocol development has focused on the use of blood derived cell types for primary somatic cell starting material given the ease of access in a clinical setting and the pre-existence of biobanked blood materials. Of additional benefit, blood derived cell types are better protected from environmental mutagens such as UV light which can lead to the accumulation of genetic variations/mutations in DNA sequence and chromosome structure.

Clonal Hematopoiesis of Indeterminant Potential (CHIP) is the age-related accumulation of somatic genetic variation(s) that confer(s) a competitive growth advantage to a distinct subpopulation of hematopoietic stem and progenitor cells relative to other stem and progenitor cells in the blood. Some of these somatic genetic variations have been associated with diseases, including bloodborne cancers (6) and cardiovascular disease, including aortic valve stenosis, venous thrombosis, and heart failure (7-10). Multiple groups have analyzed DNA sequence results from thousands of patients enrolled in large genome-wide association studies to better understand the prevalence of CHIP associated sequence variation in various populations. In a study utilizing whole exome sequencing data sets derived from 17,182 people in 22 GWAS cohorts (7), CHIP somatic sequence variations/mutations were very rare in patients younger than 40 years of age but rose in frequency in each subsequent decade of life. CHIP related sequence variations were found in 5.6% of persons 60 to 69 years of age, 9.5% of persons 70 to 79 years of age, 11.7% of persons 80 to 89 years of age, and 18.4% of persons 90 years of age or older.

In another study analyzing data from whole-exome sequencing of DNA in peripheral-blood cells from 12,380 persons, unselected for cancer or hematologic phenotypes, CHIP somatic sequence variations were observed in 0.9% of participants younger than 50 years of age but in 10.4% of those older than 65 years of age. (6). When ultra-sensitive sequencing assays (˜100× more so than whole exome sequencing) are performed, CHIP somatic sequence variations are much more common than previously appreciated (11). With this technique, 7% of individuals had CHIP related sequence variations with variant allele frequency (VAF)>10%, 39% with VAF>1%, and nearly all patients (92%) with VAF>0.1%. Furthermore, a study of healthy volunteers using a VAF lower limit of 0.03% detected by error-corrected targeted sequencing found that 95% of individuals between the ages of 50 and 70 harbor CHIP somatic sequence variations (12).

Changes to DNA structure or somatic structural variations/alterations to chromosomes associated with CHIP are less well studied but have also been associated with increased disease risk (13-14). In addition, works published in 2020 both note the relationship of increasing percentage of individuals affected with increasing age as well as slightly increased rates of this form of CHIP in males versus females. A study utilizing UK BioBank datasets noted that <1.8% of those under the age of 45 had a somatic structural variation, increasing up to 6% for males and 4.9% of females over the age of 65 (14). An equivalent study carried out on samples in the Blood Bank of Japan (BBJ) found that <4% of those individuals under the age of 30 had somatic structural variation that increased to 24.7% and 17.6% respectively for males and females between the ages of 60-69, and ultimately finding that for those over the age of 90, 40.7% of males and 31.5% of females had a somatic structural variation (15).

In view of the fact that:
1) the vast majority of cells and cell and gene therapy products are developed from cell types derived from blood (e.g., HSCs, T-cells, and primary cells for iPSC generation) and/or are delivered to the blood compartment for treatment of disease;
2) genetic variations associated with CHIP uniquely accumulate and expand in the DNA of blood cells as individuals age and are linked to an increased risk for blood cancer, cardiovascular disease (CVD) and all-cause mortality (6-7);
3) genetic variations in TP53 and KRAS genes which are associated with CHIP are also linked with solid tumor formation in other tissues (16); 4) genetic variations associated with CHIP have proven to be transmissible during stem cell transplant (17-19);
5) the manufacturing processes required for the generation of iPSC-derived therapeutic cell products accumulate genetic variation (20-24); and
6) the current common standard for assessing genetic quality and integrity of cell and gene therapy products relies on macromolecular assessment for genetic abnormalities via karyotyping and FISH that are not able to detect smaller sequence and structure-based genetic changes (25),
   there exists a definitive need to identify and remove these genetic variations from cell therapy manufacturing processes and the materials that result from them, thereby inherently reducing the risk and unknown impact of disease associated genetic variations on the health of an individual receiving a therapy.

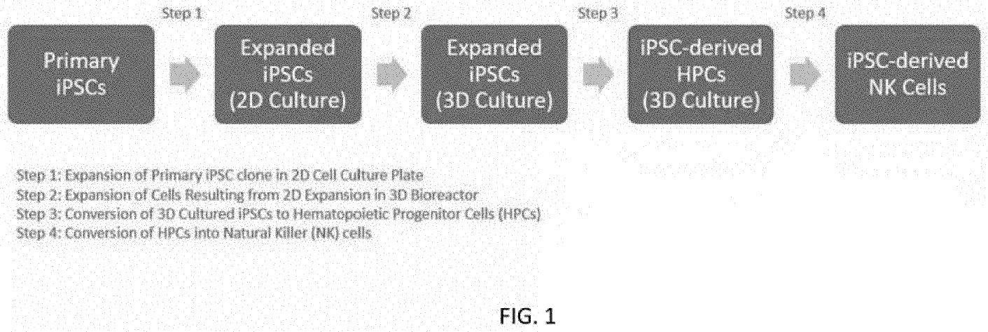

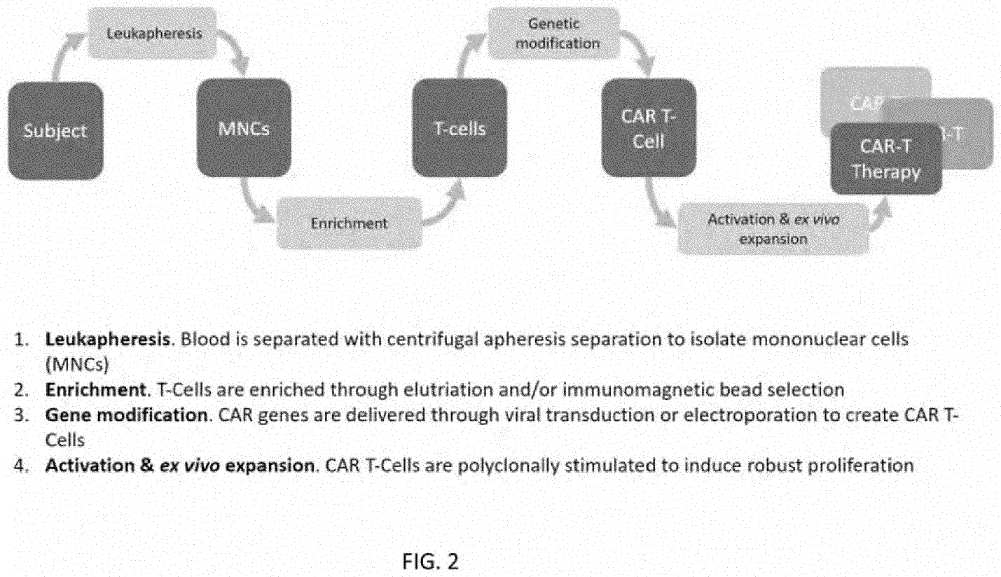

In this study the impact of processes and protocols required to manufacture cellular therapeutic products for delivery to human subjects on the accumulation and expansion of transmissible, disease-causing somatic genetic variations (sequence and structure-based) has been evaluated. To assess the impact of iPSC-related manufacturing processes and materials both a full manufacturing workflow (from primary iPSC to therapeutic NK cell product—FIG. 1), as well as independent steps/stages required to deliver iPSC derived cell therapies (cellular reprogramming to iPSCs and iPSC expansion), have been evaluated. In addition, current commercially available iPSCs have been evaluated for the presence of these genetic variations as these cell lines are oftentimes used to develop manufacturing protocols. For assessing blood stem cell manufacturing processes and materials impact, a primary CD34(+) ex vivo expansion protocol is evaluated. And for the assessment of CAR-T manufacturing processes and materials impact; a standard T-cell workflow (FIG. 2), absent the genetic engineering step, is evaluated.

### Methods

Cell Samples for iPSC Study

Samples were curated through both active collaborations and procurement through commercial vendors such as ATCC, Alstem and Applied StemCell (Table 1). Cell samples for the evaluation of a cell manufacturing workflow (iPSC→iPSC-derived NK cells) were delivered in a cryopreserved cell state (10% DMSO). The samples included in the study were from three independent manufacturing runs under a common protocol, each with different primary iPSC line starting material. The samples were collected at five different points in the manufacturing process (FIG. 1): (1) Primary iPSCs—starting material; (2) Expanded iPSCs—conventional 2D cell culture; (3) Expanded iPSCs—3D/bioreactor cell culture; (4) iPSC-derived HPCs—3D/bioreactor cell culture; and (5) iPSC-derived NK cells—final cell product.

For the assessment of commercially available human iPSC lines, samples were procured through commercial vendors and delivered in a cryopreserved state (10% DMSO). Cell samples received from collaborators to evaluate impact of the cellular reprogramming process (PBMCs+iPSCs) and iPSC expansion (primary iPSCs vs expanded iPSCs) were delivered as either cryopreserved cell samples or cell pellets frozen down and stored at −80C.

TABLE 1
iPSC Study Samples

| Commercial | Primary Somatic Cell | | | Passage | Reprogramming |
|---|---|---|---|---|---|
| iPSC Lines | Source (Human) | Gender | Age | Number | System |
| iPS01 | foreskin fibroblasts | Male | Newborn | p4-5 | Retrovirus |
| iPS15 | PBMCs | Male | adult | p4-5 | Episomal DNA |
| ASE-9215 | PBMCs | Male | 21 | NA | NA |
| ASE-9209 | dermal fibroblasts | Female | 47 | p16 | Episomal DNA |
| ASE-9101 | foreskin fibroblasts | Male | Newborn | NA | Retrovirus |
| ACS-1031 | bone marrow CD34+ cells | Female | 27 | p27 | Sendai virus |
| ACS-1030 | bone marrow CD34+ cells | Female | 31 | p37 | Sendai virus |
| ACS-1029 | bone marrow CD34+ cells | Female | 24 | p21 | Sendai virus |
| ACS-1028 | bone marrow CD34+ cells | Female | 31 | p26 | Sendai virus |
| ACS-1027 | bone marrow CD34+ cells | Male | 45 | p19 | Sendai virus |
| ACS-1026 | bone marrow CD34+ cells | Male | 31 | p33 | Sendai virus |
| ACS-1025 | bone marrow CD34+ cells | Male | 24 | p35 | Sendai virus |
| ACS-1024 | bone marrow CD34+ cells | Male | 33 | p24 | Sendai virus |
| ACS-1023 | dermal fibroblasts | Female | 36 | p39 | Retrovirus |
| ACS-1021 | cardiac fibroblasts | Male | 72 | p23 | Sendai virus |
| ACS-1013 | dermal fibroblasts | Male | 63 | p10 | Sendai virus |
| ACS-1014 | dermal fibroblasts | Male | 63 | p19 | Retrovirus |
| ACS-1012 | dermal fibroblasts | Male | 63 | p11 | Retrovirus |

| iPSC Lines for | Primary Somatic Cell | | | Passage | Reprogramming |
|---|---|---|---|---|---|
| Workflow | Source | Gender | Age | Number | System |
| Clone 1 | foreskin fibroblasts | Male | Newborn | NA | mRNA |
| Clone 2 | CD34+ Cells | NA | NA | NA | Episomal DNA |
| Clone 3 | PBMCs | NA | NA | NA | Sendai virus |

| iPSC Lines for | | | | | |
|---|---|---|---|---|---|
| Reprogramming | Primary Somatic Cell | | | Passage | Reprogramming |
| Study | Source | Gender | Age | Number | System |
| iPSC-4 | PBMC | Male | 41 | p10 | Sendai virus |
| iPSC-5 | PBMC | Male | 30 | p7 | Sendai virus |
| iPSC-6 | PBMC | Female | 25 | p7 | Sendai virus |

| iPSC Lines for | Primary Somatic Cell | | | Passage | Reprogramming |
|---|---|---|---|---|---|
| Expansion Study | Source | Gender | Age | Number | System |
| CQ-001 (later | Umbilical cord | Female | Newborn | p28 | Sendai virus |
| passage of | mononuclear cells | | | | |
| CQ-004) | | | | | |
| CQ-002 | Umbilical cord | Female | Newborn | p31 | Sendai virus |
| | mononuclear cells | | | | |
| CQ-003 (earlier | Umbilical cord | Female | Newborn | p16 | Sendai virus |
| passage of | mononuclear cells | | | | |
| CQ-005) | | | | | |
| CQ-004 (earlier | Umbilical cord | Female | Newborn | p17 | Sendai virus |
| passage of | mononuclear cells | | | | |
| CQ-001) | | | | | |
| CQ-005 (later | Umbilical cord | Female | Newborn | p18 | Sendai virus |
| passage of | mononuclear cells | | | | |
| CQ-003) | | | | | |

### Cell Samples for CD34(+) Expansion Study

Primary mobilized CD34(+) samples were procured from HemaCare and BioIVT. All samples were received in a cryopreserved cell state (10% DMSO). One vial each of Mob Cryo CD34(+)/GCSF (HemaCare) or HLA typed CD34(+) Cells (BioIVT) samples (Table 2) were retained for pre-CD34(+) expansion analysis. In parallel, a matched vial for each mobilized CD34(+) cell sample was expanded in StemSpan™ SFEM II cell culture media (Stem Cell Technologies Cat #9655) supplemented with StemSpan™ CD34+ Expansion Supplement (10×) (Stem Cell Technologies Cat #2691) for 7 days. On Day 7, five million cells were cryopreserved in 10% DMSO for subsequent post-CD34(+) expansion analysis. Total cell count, cell viability and CD34 marker expression were assessed post-thaw and on Day 7.

TABLE 2
CD34(+) Study Samples

| Donor ID | Cell Type | Gender | Age |
|---|---|---|---|
| 150081 | Mob Cryo CD34+/GCSF | Male | 58 |
| 150081 | CD34 Cells (Expanded Day 7) | Male | 58 |
| 151021 | Mob Cryo CD34+/GCSF | Male | 48 |
| 151021 | CD34 Cells (Expanded Day 7) | Male | 48 |
| 159885 | Mob Cryo CD34+/GCSF | Male | 55 |
| 159885 | CD34 Cells (Expanded Day 7) | Male | 55 |
| 148584 | Mob Cryo CD34+/GCSF | Male | 45 |
| 148584 | CD34 Cells (Expanded Day 7) | Male | 45 |
| D327292 | Mob Cryo CD34+/GCSF | Male | 58 |
| D327292 | CD34 Cells (Expanded Day 7) | Male | 58 |
| RG2193 | HLA typed CD34+ Cells/Mobilized | Male | 58 |
| RG2193 | CD34 Cells (Expanded Day 7) | Male | 58 |

### Cell Samples for T-Cell Study

8 HLA-typed PBMC samples were procured from BioIVT to assess CAR-T manufacturing workflow—isolation, activation and expansion of T-cells (FIG. 2). All eight samples were received in cryopreserved cell state (10% DMSO). One vial for each of the eight PBMC samples (Table 3) was retained for pre-isolation, activation and expansion analysis. In parallel, a matched vial for each of the eight PBMC cell samples was thawed and enriched for CD3(+) T-cells using the EasySep™ Human T Cell Enrichment Kit (Stem Cell Technologies Cat #19051). Subsequently each independent sample was then plated for activation with ImmunoCult™ Human CD3/CD28/CD2 T Cell Activator (Stem Cell Technologies Cat #10970) and expansion in ImmunoCult-XF T Cell Expansion Medium (Stem Cell Technologies Cat #10981) supplemented with Human Recombinant IL-2 (Stem Cell Technologies Cat #78036.3) for 8 days. On Day 8, five million cells were cryopreserved in 10% DMSO for subsequent post-isolation, activation and expansion analysis. Total cell count, cell viability and CD3, CD4, CD8, and HLA DR T-cell marker expression were assessed post-thaw and on Day 8. For the additive assessment of other commercially available T-cells (CD4 and CD8) and PBMCs (not subjected to isolation, activation and expansion protocols) samples were procured from BioIVT and delivered in a cryopreserved state (10% DMSO).

TABLE 3
T-cell Study Samples

| Donor ID | Cell Type | Gender | Age |
|---|---|---|---|
| 84124 | HLA typed CD8+ (T Cells) | Male | 49 |
| 84124 | HLA typed PBMCs | Male | 49 |
| 84124 | T-Cells (Enriched & Expanded Day 8) | Male | 49 |
| CC00025 | HLA typed CD4+ (T Cells) | Female | 62 |
| CC00025 | HLA typed CD8+ (T Cells) | Female | 62 |
| CC00025 | HLA typed PBMCs | Female | 60 |
| CC00025 | T-Cells (Enriched & Expanded Day 8) | Female | 60 |
| CC00061 | HLA typed CD4+ (T Cells) | Male | 56 |
| CC00061 | HLA typed PBMCs | Male | 58 |
| CC00061 | T-Cells (Enriched & Expanded Day 8) | Male | 58 |
| CC00152 | HLA typed CD4+ (T Cells) | Male | 46 |
| CC00152 | HLA typed CD8+ (T Cells) | Male | 46 |
| CC00641 | HLA typed CD8+ (T Cells) | Male | 62 |
| CC00641 | HLA typed PBMCs | Male | 62 |
| CC00641 | T-Cells (Enriched & Expanded Day 8) | Male | 62 |
| M4630 | HLA typed CD4+ (T Cells) | Male | 51 |
| M4630 | HLA typed PBMCs | Male | 50 |
| M4630 | T-Cells (Enriched & Expanded Day 8) | Male | 50 |
| M6541 | HLA typed CD8+ (T Cells) | Male | 66 |
| M6541 | HLA typed PBMCs | Male | 64 |
| M6541 | T-Cells (Enriched & Expanded Day 8) | Male | 64 |
| M7015 | HLA typed CD4+ (T Cells) | Male | 45 |
| M7015 | HLA typed CD8+ (T Cells) | Male | 47 |
| M7015 | HLA typed PBMCs | Male | 47 |
| RG1163 | HLA typed CD4+ (T Cells) | Male | 53 |
| RG1163 | HLA typed PBMCs | Male | 53 |
| RG1163 | T-Cells (Enriched & Expanded Day 8) | Male | 53 |
| RG1188 | HLA typed CD8+ (T Cells) | Male | 48 |
| RG1188 | HLA typed PBMCs | Male | 50 |
| RG1729 | HLA typed CD8+ (T Cells) | Male | 26 |
| RG1729 | HLA typed Memory CD4 | Male | 26 |
| RG1729 | HLA typed PBMCs | Male | 26 |
| RG1729 | T-Cells (Enriched & Expanded Day 8) | Male | 26 |

### Molecular Analysis

Genomic DNA (gDNA) was extracted from the cryopreserved PBMC and frozen cell pellets using the PerkinElmer Chemagic 360 B5K automated extraction platform. For targeted sequencing extracted gDNA was normalized and aliquoted to 25 ng/ul in 25 ul total volume. Custom DNA target sequencing libraries were constructed using Agilent SureSelect chemistry. The resulting libraries were analyzed on the Agilent 4200 TapeStation System and quantified by KAPA qPCR. Libraries were sequenced using the Illumina MiSeq platform with an average sequencing coverage >1000x across amplicons. Sequence reads were aligned with bwa and VCF files with sequence variants were called using Mutect2 using readily available GATK pipeline following best practices. Variants were annotated against GenBank gene models and the ClinVar database. Both somatic and germline coding variants from genes DNMT3A, TET2, ASXL1, PPM1D, JAK2, TP53, SF3B1, SRSF2, and KRAS were extracted from the annotated VCF.

A targeted sequencing panel consists of 9 coding gene targets associated with CHIP, covering the following regions: DNMT3A, TET2, TP53, ASXL1, JAK2, KRAS, PPM1D, SF3B1, and SFRS2. In addition, 129 targeted regions were identified: 3 amplicons in SRSF2; 8 amplicons in PPM1D; 13 amplicons in KRAS; 13 amplicons in TP53; 14 amplicons in ASXL1; 20 amplicons in TET2; 23 amplicons in SF3B1; 26 amplicons in DNMT3A; and 27 amplicons in JAK2.

For microarray analysis, extracted gDNA was normalized and aliquoted to 25 ng/ul in 50 ul total volume. Normalized and aliquoted gDNA samples were run on the Illumina Infinium™ Global Screening Array (GSA)—24 v2.0 BeadChip in the labs of Diagnomics (San Diego, Calif. USA). Resulting intensity IDAT files were first converted to GTC files using the Illumina Array Analysis Platform and then a VCF file with genotype and intensity information was generated using the gtc2vcf BCFtools plugin. Haplotype information was inferred from genotypes using the SHAPEIT4 software applied to a total of 547 samples genotyped in-house and 3,202 samples from the 1000 Genomes project. Mosaic chromosomal alterations were inferred from phased probe intensities using the MoChA BCFtools plugin following default settings of the MoChA pipeline. Low quality samples and samples with evidence of contaminations were excluded from the final report.

### Results

iPSC Analysis

The iPSC study was designed to assess the impact of the necessary steps/methods and cellular materials required to manufacture an iPSC-derived cell therapy product on the accumulation and expansion of somatic genetic variations (sequence and structural) associated with CHIP and disease risk. In this study three different sets of samples were evaluated:
1) cell products from key steps/stages in a complete cell therapy manufacturing workflow (Primary iPSC→iPSC-derived therapeutic cell type);
2) current commercially available iPSC lines; and
3) cell materials pre- and post- the key core methods required to manufacture an iPSC-derived cell therapy product—cellular reprogramming and iPSC expansion.
   A summary of results for each set of samples is provided below.
   iPSC-derived NK Cell Manufacturing Workflow

Fourteen samples were assayed for the existence of somatic genetic variation in both chromosomal structure (Table 4) as well as DNA sequence (Table 5) in the three independent manufacturing workflows evaluated. While no somatic sequence or structure-based variations persisted through the workflow to the endpoint iPSC-derived NK cell product for clone #1 (fibroblast+mRNA iPSCs) there were notable structural variations accumulated and subsequently lost, including a chromosome 1q gain at 8.7% of the total cell fraction that has been associated with conversion to the iPSC state. However, clones #2 and #3 both accumulated during process and carried unique somatic genetic variations through endpoint iPSC-derived NK cell product. Specifically, clone #2 accumulated a nearly 43 million nucleotide Ch.18q CN-LOH event post-3D cell culture expansion, expanding from −2% of total cell fraction to ˜11% as the culture was differentiated to hematopoietic progenitor cells (HPCs) and endpoint NK cell product. This Ch.18q locus includes genes associated with embryonic development (SALL3), cell proliferation and apoptosis (BCL2, SMADs 2, 4 and 7), T-cell activation and cytotoxicity (NFATC1 and CD226), as well as cell adhesion (CDH7, 19 and 20). Also detected in clone #2, a significant Ch.20q gain event that may be constitutional in nature given it was detected at close to 100% total cell fraction in all cell samples assayed. This significant structural variation includes the BCL2L1 gene associated with enhanced cell survival and proliferation, which suggests tumorigenic potential (26-31) and could have originated during the reprogramming process for clone #2 (CD34+ cells+Episomal iPSCs). Notably, it is detected in greater than 20% of iPSC lines (24). Clone #3 (PBMC+Sendai iPSCs) carried but lost a somatic structural Ch.12q loss from Primary iPSC to 3D Expanded iPSCs. Of greatest significance for Clone #3 is the accumulation and subsequent expansion of a pathogenic TP53 sequence-based variation linked to Li-Fraumeni Syndrome. The variation was present in ˜80% of the total cell fraction starting at the 3D Expanded iPSC stage and expanded to >90% at the HPC and iPSC-derived NK endpoint cell product stages. Genetic mutation/variation in TP53 is linked to tumorigenesis in multiple tissue types (16).

TABLE 4
Accumulated Structural Variation in an iPSC-based Cell Therapy Manufacturing Workflow

| Clone | Primary | 2D Expanded | 3D Expanded | iPSC-derived | iPSC-derived |
|---|---|---|---|---|---|
| # | iPSCs (% CF) | iPSCs (% CF) | iPSCs (% CF) | HPCs (% CF) | NKs (% CF) |
| 1 | Ch. 12q Loss | None Detected | Ch. 12q Loss | Ch. 1q Gain | None Detected |
| | (7.8%) | | (11.5%) | (8.7%) | |
| | | | Ch. 16p Loss | Ch. 10p Loss | |
| | | | (14.4%) | (9.8%) | |
| | | | Ch. 16p Loss | | |
| | | | (8.8%) | | |
| 2 | Ch. 20q Gain | Sample Not | Ch. 12q Loss | Ch. 18q CN-LOH | Ch. 18q CN-LOH |
| | (97.4%) | Available | (8.8%) | (~11%) | (~11%) |
| | | | Ch. 18q CN-LOH | Ch. 20q Gain | Ch. 20q Gain |
| | | | (~2%) | (96.8%) | (100%) |
| | | | Ch. 20q Gain | | |
| | | | (100%) | | |
| 3 | Ch. 12q Loss | Ch. 12q Loss | Ch. 12q Loss | None Detected | None Detected |
| | (9.2%) | (11.5%) | (8.2%) | | |

Ch = chromosome
CF = cell fraction

TABLE 5
Prevalence of Accumulated Sequence Variation in
an iPSC-based Cell Therapy Manufacturing Workflow

| Clone | Primary | 2D Expanded | 3D Expanded | iPSC-derived | iPSC-derived |
|---|---|---|---|---|---|
| # | iPSCs (% CF) | iPSCs (% CF) | iPSCs (% CF) | HPCs (% CF) | NKs (% CF) |
| 1 | None Detected | None Detected | None Detected | None Detected | None Detected |
| 2 | None Detected | Sample Not | None Detected | TP53: Ch. 17 | None Detected |
| | | Available | | 7675095 (C→A) | |
| | | | | Missense | |
| | | | | (6.6%) | |
| 3 | ASXL1: Ch. 20 | None Detected | TP53: Ch. 17 | TP53: Ch. 17 | TP53: Ch. 17 |
| | 3245930 (G→A) | | 7674220 (C→T) | 7674220 (C→T) | 7674220 (C→T) |
| | Missense | | Missense | Missense | Missense |
| | (0.87%) | | (79.9%) | (93.4%) | (90.2%) |

Ch = chromosome
CF = cell fraction

Commercially Available iPSC Lines

Eighteen commercially available iPSC lines generated from multiple cell types using multiple reprogramming systems, were assayed for the existence of somatic genetic variation in both chromosomal structure as well as DNA sequence (Table 6). Four of the eighteen lines had somatic sequence variations, notably in ASXL1, DNMT3A and TET2 (2×) which are associated with CHIP and with increased risk for CVD when present in blood cell DNA. As such, these would need to be evaluated prior to use in cell therapies developed for delivery into blood. Eight of the eighteen lines had somatic structural variations, with one of the lines (ACS-1029) in combination with a TET2 missense mutation. Notably, two iPSC lines, iPS15 (PBMC+episomal iPSCs) and ACS-1030 (BM+Sendai iPSCs) both had an aforementioned Ch.20q gain at 3.9% and 45.1% total cell fraction, respectively. With extended culture it is highly likely that the 20q gain event will be detected at higher and higher cell fractions in these iPSC lines given its association with enhanced proliferation, survival and tumorigenesis (27-32). Additionally, two independent, yet both significant Ch.14q loss events were detected at 95.1% and 76.2% total cell fraction accordingly in ASE-9215 (derived from PBMCs) iPSC line. Ch.14q loss events have been associated with increased risk (OR=115) for Chronic Lymphocytic Leukemia (CLL) (14). Both aforementioned lines with somatic structural variants would not be acceptable/useable starting material for manufacturing of a cell therapy product as they confer an increase in cancer risk, notably for CLL if the therapy were to be delivered to the blood compartment. In addition, three lines had focal deletions at 12q, 16p, and 16q for loss of TMEM132D, RBFOX1 and WWOX respectively. The latter which spans a common chromosomal fragile site and appears to function as a tumor suppressor gene (33). In total, 11/18 (˜61%) commercially available iPSC lines evaluated carry genetic variations at total cell fractions worthy of review for downstream use in the manufacturing of cell therapies—four lines with structural variations associated with tumorigenesis and cancer and four with sequence variations associated with CHIP.

TABLE 6
Commercially Available iPSC Lines
with Accumulated Genetic Variations

| Commercial | Sequence | Structure |
|---|---|---|
| iPSC Lines | Variation (% CF) | Variation (% CF) |
| iPS01 | | |
| iPS15 (PBMC) | | Ch. 20p Loss (4.1%)* |
| | | Ch. 20q Gain (3.9%)* |
| ASE-9215 | | Ch. 14q Loss (95.1%)* |
| (PBMC) | | Ch. 14q Loss (76.2%)* |
| ASE-9209 | ASXL1: Ch. 20 32358788 | |
| (Fibroblast) | (CAGA→C) del (46.8%) | |
| ASE-9101 | | Ch. 16p Loss (13.6%) |
| (Neonatal | | (RBFOX1) |
| Fibroblast) | | |
| ACS-1031 | | |
| ACS-1030 | | Ch. 20q Gain (45.1%) |
| (BM CD34+) | | (BCL2.1L) |
| | | Ch. 1q Gain (1.6%) |
| | | Ch. 12q Loss (11.6%) |
| | | (TMEM132D) |
| ACS-1029 | TET2: Ch. 4 105243685 | Ch12q Loss (8.4%) |
| (BM CD34+) | (C→G) Missense (96.6%) | (TMEM132D) |
| | | Ch. 16p Loss (12.0%) |
| | | (RBFOX1) |
| ACS-1028 | | Ch. 5p Loss (7.5%) |
| | | (TERT) |
| | | Ch. 16p Loss (9.5%) |
| | | (RBFOX1) |
| ACS-1027 | | |
| ACS-1026 | | |
| ACS-1025 | | |
| ACS-1024 | DNMT3A: Ch. 2 25300218 | |
| (BM CD34+) | (C→T) Missense (97.8%) | |
| ACS-1023 | TET2: Ch. 4 105235666 | |
| (Fibroblast) | (C→T) Missense (100%) | |
| ACS-1021 | | Ch. 15q CN-LOH (2.7%) |
| ACS-1013 | | |
| ACS-1014 | | Ch. 5p Loss (8.5%) |
| (Fibroblast) | | Ch. 16p Loss (11.9%) |
| | | (RBFOX1) |
| | | Ch. 16q Loss (9.2%) |
| | | (WWOX) |
| ACS-1012 | | |

Ch = chromosome
CF = cell fraction

Cellular Reprogramming and iPSC Expansion

Early results from the sequence analysis of both the three matched pairs of PBMC primary samples and subsequently derived iPSC lines for evaluation of impact of cellular reprogramming, as well as the samples to evaluate impact of iPSC expansion, revealed no somatic sequence variations. Given the high frequency of somatic structural variations residing in the analysis of commercially available iPSC lines and the potential to accumulate these types of variations at different steps in the manufacturing process as evidenced in the iPSC-NK cell workflow data, it is anticipated that new somatic structural variants will be picked up from both the cellular reprogramming and iPSC expansion samples currently in analysis.

### CD34+ Cell Expansion

Early results from sequence analysis of the six pre-expansion CD34+ cell samples identified one Mob Cryo CD34+/GCSF cell sample with a germline ASXL1 sequence variant associated with CHIP. Given that the age of donors for the six samples included in the CD34+ cell expansion study is 45 years and up and that somatic genetic variations associated with CHIP increase with age, it is expected that some of the samples will have somatic structural variations. Pending data from the analysis of these six samples post-expansion will provide key insight into the impact of ex vivo expansion protocol on the accumulation of new somatic genetic variations as well the expansion of pre-existing ones.

### T-Cell Isolation, Activation and Expansion

Early results from sequence analysis of the eight pre-isolation, activation and expansion PBMC samples identified a novel pathogenic DNMT3A somatic sequence variant at 2.6% total cell fraction notably associated with CHIP and increased risk for acute myeloid leukemia (AML) in the sample from Donor ID 84124. Of interest, the matching CD8+ T-cell sample that was isolated in parallel to the PBMC sample for this donor to test for persistence of somatic genetic variation had the same DNMT3A pathogenic variant at 2.8% total cell fraction. In addition, while the pre-isolation, activation and expansion PBMC sample from Donor ID CC00152 did not have a somatic sequence variation, the matching CD8+ T-cell sample that was isolated in parallel did have TET2 missense mutation associated with CHIP at 3.9% of total cell fraction demonstrating that these variations can be accumulated during in vivo conversion to T-cell populations. Pending data from the analysis of these eight samples post-isolation, activation and expansion will provide key insight into the impact of manufacturing protocols required for CAR-T cellular therapies on the accumulation of new somatic genetic variations as well the expansion of pre-existing ones.

### Discussion

Cell and gene therapy as an industry is developing at a rapid pace, for example, in 2020 there were over 1,000 companies worldwide developing therapies, over 1,000 clinical trials in progress and over 15 billion dollars in financing supporting the industry (alliancenn.org/wp-content/uploads/2020/08/ARM_1H-Report_-FINAL.pdf). Of note, significant progress has been made in advancing therapies derived from pluripotent stem cells (PSCs) to treat disorders like Parkinson's Disease, Diabetes, Macular Degeneration and bloodborne cancers like BCL, CLL, AML and multiple myeloma. The manufacturing of these PSC-derived therapies oftentimes requires generation, expansion, genetic manipulation and differentiation of PSCs to achieve a useful endpoint cell product for therapy—all steps of which can lead to the accumulation of changes to DNA sequences and chromosome structures. These changes of course also oftentimes come with an increased risk for disease. Of note, the manufacturing process for iPSC-derived therapies utilizes cells derived from blood and subsequently generated therapies are delivered back into the blood. Likewise, blood stem cell transplants and CAR-T therapies rely on accumulating source material from the blood to ex vivo manipulate (e.g., expand, engineer, differentiate) and return to the blood compartment for therapeutic intervention. CHIP itself is an age-related phenomenon whereby disease risk associated changes in DNA sequences and chromosomal structures uniquely accumulate and expand in the DNA of blood cells. Given that these cells and cell and gene therapy manufacturing processes utilize blood cell types to treat disease, that somatic genetic changes associated with disease risk that are transmissible to recipient during transplant accumulate, and that current standards used to assess genetic integrity and genetic variation oftentimes fail to pick up genetic variations accumulated in small fractions of the cells assayed, it is necessary to screen the primary starting materials and therapeutic cell products resulting from these manufacturing workflows with sensitive genetic assays that can pick up both somatic sequences, as well as structural-based changes.

The results of the iPSC study demonstrate that manufacturing processes and cellular materials required to generate these PSC-based cell therapies carry and accumulate significant genetic variations associated with disease, regardless of cell donor age, primary cell type, and reprogramming system used to generate iPSCs. Genetic assays and analysis pipelines employed in the study detected both sequence and structure-based genetic variations associated with disease risk. Notably, the analysis of a consistent manufacturing workflow on three different starting iPSC lines identified unique variations associated with tumorigenic potential (TP53 and Ch.20q gain) that ended up in the final cell product in two of the three workflows. Additionally, the TP53 sequence variant in Clone #3, as well as the 18q CN-LOH structural variant in Clone #2, both originated during the 3D expansion step and persisted through to endpoint cell product. Of note, the workflow analyzed was for development of a blood specific cell type, while the two workflows that ultimately failed to deliver endpoint therapeutic cells free of tumorigenic potential utilized iPSCs that were derived from blood cell types. Likewise, 8 out of the commercially available 18 iPSC lines carried significant disease risk associated with genetic variation, of which 5 were derived from blood cell types and 5 carried somatic variations associated with CHIP. As such it is believed that the identification of somatic sequences and structural-based variations in primary cell materials for the generation of iPSCs, (e.g., iPSCs themselves, as well as endpoint PSC-derived therapeutic cell products) is necessary to ensure that cell therapy products are free of these variations and associated risks before delivery to a patient. In addition, it is believed that the detection of and monitoring for these somatic genetic variations during cellular manufacturing workflows will lead to the development of more consistent, higher genetic quality and as a result more cost-effective cell therapy product. It is anticipated that pending data from the analysis of pre- and post-expansion of CD34+ cells as well as pre- and post-isolation, activation and expansion of T-cells from primary PBMC cell populations will provide additional support and extend the need for this type of monitoring into blood stem cell and CAR-T manufacturing processes for cellular therapy products, notably cell expansion protocols.

### REFERENCES

1) Granot N, Storb R. History of hematopoietic cell transplantation: challenges and progress. Haematologica. 2020 Dec. 1; 105(12):2716-2729. doi: 10.3324/haematoL2019.245688.
2) Vormittag P, Gunn R, Ghorashian S, Veraitch F S. A guide to manufacturing CAR T cell therapies. Curr Opin Biotechnol. 2018 October; 53:164-181.
3) Goldsmith, S. R., Ghobadi, A. and DiPersio, J. F., 2020. Hematopoeitic Cell Transplantation and CAR T-Cell Therapy: Complements or Competitors?. Frontiers in Oncology 10:2904.
4) Thomson, J. A., Itskovitz-Eldor, J., Shapiro, S. S., Waknitz, M. A., Swiergiel, J. J., Marshall, V. S. and Jones, J. M., 1998. Embryonic stem cell lines derived from human blastocysts. science 282:1145-1147.
5) Takahashi, K., Tanabe, K., Ohnuki, M., Narita, M., Ichisaka, T., Tomoda, K. and Yamanaka, S., 2007. Induction of pluripotent stem cells from adult human fibroblasts by defined factors. cell 131:861-872.
6) Yu, J., Vodyanik, M. A., Smuga-Otto, K., Antosiewicz-Bourget, J., Frane, J. L., Tian, S., Nie, J., Jonsdottir, G. A., Ruotti, V., Stewart, R. and Slukvin, I. I., 2007. Induced pluripotent stem cell lines derived from human somatic cells. science 318:1917-1920.
7) Genovese G, Kähler A K, Handsaker R E, Lindberg J, Rose S A, Bakhoum S F, Chambert K, Mick E, Neale B M, Fromer M, Purcell S M, Svantesson O, Landén M, Höglund M, Lehmann S, Gabriel S B, Moran J L, Lander E S, Sullivan P F, Sklar P, Grönberg H, Hultman C M, McCarroll S A. Clonal hematopoiesis and blood-cancer risk inferred from blood DNA sequence. N Engl J Med. 2014 Dec. 25; 371(26):2477-87.
8) Jaiswal, S., P. Fontanillas, J. Flannick, A. Manning, P. V. Grauman, B. G. Mar, R. C. Lindsley, C. H. Mermel, N. Burtt, A. Chavez, J. M. Higgins, V. Moltchanov, F. C. Kuo, M. J. Kluk, B. Henderson, L. Kinnunen, H. A. Koistinen, C. Ladenvall, G. Getz, A. Correa, B. F. Banahan, S. Gabriel, S. Kathiresan, H. M. Stringham, M. I. McCarthy, M. Boehnke, J. Tuomilehto, C. Haiman, L. Groop, G. Atzmon, J. G. Wilson, D. Neuberg, D. Altshuler, and B. L. Ebert. 2014. Age-Related Clonal Hematopoiesis Associated with Adverse Outcomes. New Engl J Medicine 371:2488-2498.
9) Mas-Peiro, S., J. Hoffmann, S. Fichtlscherer, L. Dorsheimer, M. A. Rieger, S. Dimmeler, M. Vasa-Nicotera, and A. M. Zeiher. 2019. Clonal haematopoiesis in patients with degenerative aortic valve stenosis undergoing transcatheter aortic valve implantation. Eur Heart J 41:933-939.
10) Sano, S., Y. Wang, Y. Yura, M. Sano, K. Oshima, Y. Yang, Y. Katanasaka, K.-D. Min, S. Matsuura, K. Ravid, G. Mohi, and K. Walsh. 2019. JAK2 V617F-Mediated Clonal Hematopoiesis Accelerates Pathological Remodeling in Murine Heart Failure. Jacc Basic Transl Sci 4: 684-697.
11) Bazeley, P., R. Morales, and W. H. W. Tang. 2020. Evidence of Clonal Hematopoiesis and Risk of Heart Failure. Curr Hear Fail Reports 17:271-276.
12) Swanton, C., Venn, O., Aravanis, A., Hubbell, E., Maddala, T., Beausang, J., Filippova, D., Gross, S., Jamshidi, A., Shen, L., Valouev, A., Zhang, N., Bolton, K., Yeatman, T., Seiden, M., Oxnard, G., Liu, M. C., Williams, R., Hartman, A., & Baselga, J. (2018). Prevalence of clonal hematopoiesis of indeterminate potential (CHIP) measured by an ultra-sensitive sequencing assay: Exploratory analysis of the Circulating Cancer Genome Atlas (CCGA) study. Journal of Clinical Oncology, 36, 12003-12003.
13) Young, A., Challen, G., Birmann, B., & Druley, T. (2016). Clonal haematopoiesis harbouring AML-associated mutations is ubiquitous in healthy adults. Nature Communications, 7.
14) Loh P R, Genovese G, Handsaker R E, Finucane H K, Reshef Y A, Palamara P F, Birrmann B M, Talkowski M E, Bakhoum S F, McCarroll S A, Price A L. Insights into clonal haematopoiesis from 8,342 mosaic chromosomal alterations. Nature. 2018 Ju1; 559(7714):350-355.
15) Loh, PR., Genovese, G. & McCarroll, S. A. 2020. Monogenic and polygenic inheritance become instruments for clonal selection. Nature 584: 136-141.
16) Terao, C., Suzuki, A., Momozawa, Y., Akiyama, M., Ishigaki, K., Yamamoto, K., Matsuda, K., Murakami, Y., McCarroll, S. A., Kubo, M. and Loh, P. R., 2020. Chromosomal alterations among age-related haematopoietic clones in Japan. Nature 584:130-135.
17) Martincorena 1, Campbell P J. Somatic mutation in cancer and normal cells. Science. 2015 Sep. 25; 349(6255):1483-9. doi: 10.1126/science.aab4082. Epub 2015 Sep. 24. Erratum in: Science. 2016 Mar. 4; 351(6277).
18) Christopher J. Gibson, James A. Kennedy, Sarah Nikiforow, Frank C. Kuo, Edwin P. Alyea, Vincent Ho, Jerome Ritz, Robert Soiffer, Joseph H. Antin, R. Coleman Lindsley; Donor-engrafted CHIP is common among stem cell transplant recipients with unexplained cytopenias. Blood 2017; 130 (1):91-94.
19) Wilk, C. Matthias; Manz, Markus G.; Boettcher, Steffen Clonal hematopoiesis in hematopoietic stem cell transplantation, Current Opinion in Hematology: March 2021; 28(2):94-100.
20) Louis Nevejan, Friedel Nollet, Helena Devos, Matthijs Vynck, Pieter Van Vlierberghe, Mercedeh Tajdar, Tom Lodewyck, Dominik Selleslag; Malignant progression of donor-engrafted clonal hematopoiesis in sibling recipients after stem cell transplantation. Blood Adv 2020; 4 (22):5631-5634.
21) Lefort, N., Feyeux, M., Bas, C., Fe′raud, O., Bennaceur-Griscelli, A., Tachdjian, G., Peschanski, M., and Perrier, A. L. (2008). Human embryonic stem cells reveal recurrent genomic instability at 20q11.21. Nat. Biotechnol. 26:1364-1366.
22) Laurent L C, Ulitsky I, Slavin I, Tran H, Schork A, Morey R, Lynch C, Harness J V, Lee S, Barrero M J, Ku S, Martynova M, Semechkin R. Galat V, Gottesfeld J, Izpisua Belmonte J C, Murry C, Keirstead H S, Park H S, Schmidt U. Laslett A L. Muller F J, Nievergelt C M/1, Shamir R, Loring J F. Dynamic changes in the copy number of pluripotency and cell proliferation genes in human ESCs and iPSCs during reprogramming and time in culture. Cell Stem Cell. 2011 Jan. 7; 8(1):106-18.
23) Merkle F T, Ghosh S, Kamitaki N, Mitchell J, Avior Y, Mello C, Kashin S. Mekhoubad S, Ilic D, Charlton M. Saphier G. Handsaker R E, Genovese G, Bar S, Benvenisty N, McCarroll S A, Eggan K. Human pluripotent stem cells recurrently acquire and expand dominant negative P53 mutations. Nature. 2017 May 11; 545(7653):229-233.
24) Matteo D'Antonio, Paola Benaglio, David Jakubosky, William W. Greenwald, Hiroko Matsui, Margaret K. R. Donovan, He Li, Erin N. Smith, Agnieszka D'Antonio-Chronowska, Kelly A. Frazer, Insights into the Mutational Burden of Human Induced Pluripotent Stem Cells from an Integrative Multi-Omics Approach, Cell Reports, 2018; 24(4):883-894.
25) Said Assou, Nicolas Girault, Mathilde Plinet, Julien Bouckenheimer, Caroline Sansac, Marion Combe, Joffrey Mianné, Chloé Bourguignon, Mathieu Fieldes, Engi Ahmed, Thérèse Commes, Anthony Boureux, Jean-Marc Lemaitre, John De Vos, Recurrent Genetic Abnormalities in Human Pluripotent Stem Cells: Definition and Routine Detection in Culture Supernatant by Targeted Droplet Digital PCR, Stem Cell Reports, 2020; 14(1):1-8.
26) Sullivan S, Stacey G N, Akazawa C. Aoyama N, Baptista R, Bedford P, Bennaceur Griscelli A, Chandra A, Elwood N, Girard M, Kawaniata S, Hanatani T, Latsis T, Lin S, Ludwig T E, Malygina T, Mack A, Mountford J C, Noggle S, Pereira L V, Price J, Sheldon M, Srivastava A, Stachelscheid H, Velayudhan S R, Ward N J, Turner M L, Barry J, Song J. Quality control guidelines for clinical-grade human induced pluripotent stem cell lines. Regen Med. 2018 October; 13(7):859-866.
27) Avery S, Hirst A J, Baker D et al. BCL-XL mediates the strong selective advantage of a 20q11.21 amplification commonly found in human embryonic stem cell cultures. Stem Cell Reports 2013; 1:379-386.
28) Kyriakides, O., Halliwell, J. A. & Andrews, P. W. Acquired genetic and epigenetic variation in human pluripotent stem cells. Adv. Biochem. Eng. Biotechnol. 163:187-206.
29) Bai, H. et al. Bcl-xL enhances single-cell survival and expansion of human embryonic stem cells without affecting self-renewal. Stem Cell Res. 8:26-37.
30) Nguyen, H. T. et al. Gain of 20q1121 in human embryonic stem cells improves cell survival by increased expression of Bcl-xL. Mol. Hum. Reprod. 20:168-177.
31) Beroukhim R, Mermel C H, Porter D, Wei G, Donovan J, Barretina J, Boehm J S, Urashima M, Henry K T M, Pinchback R M et al. The landscape of somatic copy-number alteration across human cancers. Nature 2010; 463:899-905.
32) Tabach Y, Kogan-Sakin I, Buganim Y, Solomon H, Goldfinger N, Hovland R, Ke X-S, Oyan A M, Kalland K-H, Rotter V et al. Amplification of the 20q chromosomal arm occurs early in tumorigenic transformation and may initiate cancer. PloS One 2011; 6.
33) Rajaram M, Zhang J, Wang T, et al. Two Distinct Categories of Focal Deleions in Cancer Genomes. PLoS One. 2013; 8(6):e66264.

What is claimed is:

1. A method of assessing quality of cells during a manufacturing process comprising
a. receiving a sample of cells at one or more time points during a manufacturing process;
b. sequencing at least part of the genome of one or more cells received at the one or more time points; and
c. identifying in the received cells a defect in one or more genes selected from the group consisting of DNMT3A, TET2, ASXL1, PPM1D, JAK2, TP53, SRSF2, KRAS and SF3B1.

2. The method of claim 1, wherein the sample of cells is received at one or more time points during the manufacturing process selected from the group consisting of: receipt of starter cells, completion of one or more stages of manipulation of the cells, and receipt of manufactured cells prior to use.

3. The method of claim 2, wherein the one or more stages of manipulation of the cells are selected from the group consisting of cellular reprogramming, culture and expansion, genetic manipulation, differentiation, harvest, heterogeneity/subtyping, cryopreservation, thawing, isolation, enrichment, single cell cloning, and purification.

4. The method of claim 3, wherein cellular reprogramming of the cells comprises converting an isolated somatic primary cell to an induced pluripotent stem cell.

5. The method of claim 3, wherein the manipulation of the cells comprises manipulating a T cell to a CAR T cell.

6. The method of claim 5, wherein the CAR T cell is engineered to target an antigen of interest on a cancer cell.

7. The method of claim 5, wherein the CAR T cell is engineered to target an antigen of interest on a tumor cell.

8. The method of claim 3, wherein the genetic manipulation comprises manipulating cells using one or more of CRISPR, TALEN, Zn-Finger, and vector delivery systems.

9. The method of claim 8, wherein the gene editing system is delivered to a cell via a vector delivery system.

10. The method of claim 9, wherein the vector delivery system is a RNA, DNA, or viral vector delivery system.

11. The method of claim 3, wherein the genetic manipulation is selected from the group consisting of correcting one or more genetic defects, reducing expression of one or more genes, and increasing expression of one or more genes.

12. The method of claim 3, wherein the genetic manipulation comprises inactivating TET2.

13. The method of claim 3, wherein differentiation comprises converting a starter cell into a therapeutic cell type.

14. The method of claim 13, wherein the starter cell is a pluripotent cell.

15. The method of claim 13, wherein the therapeutic cell type is selected from the group consisting of beta cells, cardiomyocytes, satellite cells, retinal cells, NK cells, and neural cells.

16. The method of claim 1, wherein one or more cells in the manufacturing process are manufactured from a population of starter cells.

17. The method of claim 16, wherein the starter cells are stem cells.

18. The method of claim 16, wherein the starter cells are pluripotent cells or somatic cells.

19. The method of claim 16, wherein the starter cells are induced pluripotent stem cells (iPSCs) or embryonic stem cells (ESCs).

20. The method of claim 16, wherein the starter cells are hematopoietic stem cells (HSCs) or T cells.

21. The method of claim 16, wherein the population of starter cells are obtained from a blood sample.

22. The method of claim 16, wherein the population of starter cells are obtained from a subject.

23. The method of claim 22, wherein the subject is a subject in need thereof.

24. The method of claim 22, wherein the subject is a donor subject.

25. The method of claim 1, wherein the defect is a sequence-based mutation.

26. The method of claim 25, wherein the sequence-based mutation is a mis-sense mutation, silent mutation, frame-shift mutation, nonsense mutation, insertion mutation, deletion mutation, or splice-site disruption.

27. The method of claim 25, wherein the defect is a somatic sequence-based mutation or a germline sequence-based mutation.

28. The method of claim 1, wherein the defect is in DNMT3A in exons 7 to 23.

29. The method of claim 1, wherein the defect is a mis-sense mutation in DNMT3A selected from the group consisting of G543C, S714C, F732C, Y735C, R736C, R749C, F751C, W753C, and L889C.

30. The method of claim 1, wherein the defect is a V617F mutation in JAK2.

31. The method of claim 1, wherein the defect is a disruptive mutation in TET2.

32. The method of claim 1, wherein the defect is a disruptive mutation in PPM1D.

33. The method of claim 1, wherein the defect is a mis-sense mutation in TP53 selected from the group consisting of R175H, G245S, R248W, R273G, P151S, R181H, H193R, M237I, G245C, R248Q, R267W, and R273L.

34. The method of claim 1, wherein the one or more genes are associated with tumorigenesis.

35. The method of claim 34, wherein the one or more genes are selected from the group consisting of TP53, KRAS, ASXL1, JAK2, SFSR2, and SFSB1.

36. The method of claim 34, wherein the one or more genes are selected from the group consisting of TP53 and KRAS.

37. The method of claim 1, wherein the one or more genes are associated with cancer.

38. The method of claim 37, wherein the one or more genes are selected from the group consisting of DNMT3A, TET2, ASXL1, PPM1D, JAK2, SF3B1, SRSF2, and TP53.

39. The method of claim 37, wherein the one or more genes are selected from the group consisting of DNMT3A, TET2, and ASXL1.

40. The method of claim 1, wherein the one or more genes are associated with blood cancer.

41. The method of claim 40, wherein the one or more genes are selected from the group consisting of TET2 and DNMT3A.

42. The method of claim 1, further comprising identifying in the received cells a defect in one or more genes selected from the group consisting of PCM1, HIF1A, and APC.

43. The method of claim 42, wherein the defect is a sequence-based mutation.

44. The method of claim 1, further comprising identifying in the received cells a defect in one or more genes selected from the group consisting of TERT and CHEK2.

45. The method of claim 1, further comprising identifying in the received cells a defect in one or more genes selected from the group consisting of CBL, KMT2C, ATM, CHEK2, KDR, MGA, DNMT3B, ARID2, SH2B3, MPL, RAD21, SRSF2, and CCND2.

46. The method of claim 1, further comprising identifying in the received cells a defect in one or more genes selected from the group consisting of HPRT, JAK1, JAK3, SLAMF6, IRF1, PLRG1, STAT3, and Notch1.

47. The method of claim 1, further comprising identifying in the received cells a structure-based mutation in one or more genes selected from the group consisting of DNMT3A, TET2, ASXL1, PPM1D, JAK2, TP53, SRSF2, KRAS and SF3B1.

48. The method of claim 47, wherein the structure-based mutation is a duplication, deletion, copy number variation, inversion, or translocation.

49. The method of claim 47, wherein the structure-based mutation occurs on one or more chromosomes selected from the group consisting of Ch2, Ch4, Ch9, Ch12, Ch17, and Ch20.

50. The method of claim 47, wherein the structure-based mutation occurs on one or more chromosomes selected from the group consisting of Ch2p23, Ch4q24, Ch20q11, Ch17q23, Ch9p24, Ch17p23, Ch17q25, Ch2q33, and Ch12p12.

51. The method of claim 1, further comprising identifying in the received cells a structure-based mutation occurring on one or more chromosomes selected from the group consisting of Ch1, Ch12, Ch17q, Ch20q11, and X-chromosome.

52. The method of claim 1, further comprising identifying in the received cells a structure-based mutation occurring on one or more chromosomes selected from the group consisting of Ch3, Ch4, Ch5, Ch7, Ch8, Ch9, Ch11, Ch12, Ch13, Ch14, and Ch18.

53. The method of claim 1, wherein the sample of cells comprises iPSCs derived from a blood sample of a subject in need of treatment.

54. The method of claim 1, wherein the sample of cells comprises iPSCs derived from a blood sample of a donor subject.

55. The method of claim 1, wherein the sample of cells comprises hematopoietic stem cells derived from a blood sample of a subject in need of treatment.

56. The method of claim 1, wherein the sample of cells comprises hematopoietic stem cells derived from a blood sample of a donor subject.

57. The method of claim 1, wherein the sample of cells comprises T cells derived from a blood sample of a subject in need of treatment.

58. The method of claim 1, wherein the sample of cells comprises T cells derived from a blood sample of a donor subject.

59. The method of claim 1, further comprising identifying one or more time points during the manufacturing process wherein a defect in the one or more genes is identified.

60. The method of claim 1, further comprising isolating a subpopulation of received cells that exhibit no identified defects in the one or more genes.

61. The method of claim 60, further comprising subjecting the isolated subpopulation of received cells to the cell therapy manufacturing process.

62. The method of claim 1, further comprising isolating a subpopulation of received cells that exhibit a defect in the one or more genes.

63. The method of claim 62, further comprising correcting the defect in the one or more genes.

64. The method of claim 63, further comprising subjecting the corrected isolated subpopulation of received cells to the cell therapy manufacturing process.

65. The method of claim 1, wherein the sample of cells is a sample of manufactured cells.

66. A method of maintaining quality of cells during a manufacturing process comprising:
a. sequencing at least part of a genome of one or more iPSC donor cells from a subject;
b. identifying in the donor cells a defect in one or more genes selected from the group consisting of DNMT3A, TET2, ASXL1, PPM1D, JAK2, TP53, SRSF2, KRAS and SF3B1;
c. isolating the donor cells that exhibit no identified defects in the one or more genes;
d. subjecting the isolated donor cells to a cell therapy manufacturing process to produce one or more manufactured cells;
e. sequencing at least part of the genome of the one or more manufactured cells;
f. identifying in the manufactured cells a defect in one or more genes selected from the group consisting of DNMT3A, TET2, ASXL1, PPM1D, JAK2, TP53, SRSF2, KRAS and SF3B1; and
g. isolating the manufactured cells that exhibit no identified defects in the one or more genes.

67. The method of claim 66, further comprising a step of sequencing at least part of the genome of the isolated donor cells during one or more stages of the cell therapy manufacturing process; identifying in the cells in the manufacturing process a defect in one or more genes selected from the group consisting of DNMT3A, TET2, ASXL1, PPM1D, JAK2, TP53, SRSF2, KRAS and SF3B1; and isolating the cells in the manufacturing process that exhibit no identified defects in the one or more genes.

68. The method of claim 67, wherein the isolated cells are subjected to one or more additional stages of the cell therapy manufacturing process.

69. The method of claim 66, further comprising a step of administering to the subject the isolated manufactured cells that exhibit no identified defects in the one or more genes.

70. The method of claim 69, wherein the isolated manufactured cells are administered to the subject to treat a disease or disorder.

71. The method of claim 70, wherein the disease or disorder is a blood, immune, metabolic, neurologic, or cardiovascular disorder.

72. A method of maintaining quality of cells during a manufacturing process comprising:
a. sequencing at least part of a genome of one or more HSC or T cell donor cells from a subject;
b. identifying in the donor cells a defect in one or more genes selected from the group consisting of DNMT3A, TET2, ASXL1, PPM1D, JAK2, TP53, SRSF2, KRAS and SF3B1;
c. isolating the donor cells that exhibit no identified defects in the one or more genes;
d. subjecting the isolated donor cells to a cell therapy manufacturing process to produce one or more manufactured cells;
e. sequencing at least part of the genome of the one or more manufactured cells;
f. identifying in the manufactured cells a defect in one or more genes selected from the group consisting of DNMT3A, TET2, ASXL1, PPM1D, JAK2, TP53, SRSF2, KRAS and SF3B1; and
g. isolating the manufactured cells that exhibit no identified defects in the one or more genes.

73. The method of claim 72, further comprising a step of sequencing at least part of the genome of the isolated donor cells during one or more stages of the cell therapy manufacturing process; identifying in the cells in the manufacturing process a defect in one or more genes selected from the group consisting of DNMT3A, TET2, ASXL1, PPM1D, JAK2, TP53, SRSF2, KRAS and SF3B1; and isolating the cells in the manufacturing process that exhibit no identified defects in the one or more genes.

74. The method of claim 73, wherein the isolated cells are subjected to one or more additional stages of the cell therapy manufacturing process.

75. The method of claim 72, further comprising a step of administering to the subject the isolated manufactured cells that exhibit no identified defects in the one or more genes.

76. The method of claim 75, wherein the isolated manufactured cells are administered to the subject to treat a disease or disorder.

77. The method of claim 76, wherein the disease or disorder is a cancer.

78. The method of claim 76, wherein the disease or disorder is selected from the group consisting of acute myeloid leukemia, acute lymphoblastic leukemia, myelodysplastic syndrome, myeloproliferative neoplasm, germ cell tumor, neuroblastoma, Ewing sarcoma, and medulloblastoma.

79. The method of claim 76, wherein the disease or disorder is a solid tumor.

80. The method of claim 79, wherein the solid tumor is a non-malignant tumor.

81. The method of claim 79, wherein the solid tumor is a malignant tumor.

82. A method of evaluating quality of cells comprising:
a. receiving a sample of somatic cells or pluripotent cells prior to a manufacturing process;
b. sequencing at least part of the genome of the somatic cells or pluripotent cells; and
c. identifying in the somatic cells or pluripotent cells a defect in one or more genes selected from the group consisting of DNMT3A, TET2, ASXL1, PPM1D, JAK2, TP53, SRSF2, KRAS and SF3B1.

83. The method of claim 82, wherein the pluripotent cells are induced pluripotent stem cells or embryonic stem cells.

84. The method of claim 82, wherein the pluripotent cells are iPSCs derived from a blood sample of a subject in need of treatment.

85. The method of claim 82, wherein the pluripotent cells are iPSCs derived from a blood sample of a donor subject.

86. The method of claim 82, wherein the defect is a sequence-based mutation.

87. The method of claim 86, wherein the sequence-based mutation is a mis-sense mutation, silent mutation, frame-shift mutation, nonsense mutation, insertion mutation, deletion mutation, or splice-site disruption.

88. The method of claim 82, wherein the defect is in DNMT3A in exons 7 to 23.

89. The method of claim 82, wherein the defect is a mis-sense mutation in DNMT3A selected from the group consisting of G543C, S714C, F732C, Y735C, R736C, R749C, F751C, W753C, and L889C.

90. The method of claim 82, wherein the defect is a V617F mutation in JAK2.

91. The method of claim 82, wherein the defect is a disruptive mutation in TET2.

92. The method of claim 82, wherein the defect is a disruptive mutation in PPM1D.

93. The method of claim 82, wherein the defect is a mis-sense mutation in TP53 selected from the group consisting of R175H, G245S, R248W, R273G, P151S, R181H, H193R, M237L, G245C, R248Q, R267W, and R273L.

94. The method of claim 82, wherein the one or more genes are associated with tumorigenesis.

95. The method of claim 94, wherein the one or more genes are selected from the group consisting of TP53, KRAS, ASXL1, JAK2, SFSR2, and SFSB1.

96. The method of claim 94, wherein the one or more genes are selected from the group consisting of TP53 and KRAS.

97. The method of claim 82, wherein the one or more genes are associated with blood cancer.

98. The method of claim 97, wherein the one or more genes are selected from the group consisting of TET2 and DNMT3A.

99. The method of claim 82, further comprising identifying in the pluripotent cells a sequence-based mutation in one or more genes selected from the group consisting of PCM1, HIF1A, and APC.

100. The method of claim 82, further comprising identifying in the pluripotent cells a structure-based mutation in one or more genes selected from the group consisting of DNMT3A, TET2, ASXL1, PPM1D, JAK2, TP53, SRSF2, KRAS and SF3B1.

101. The method of claim 100, wherein the structure-based mutation is a duplication, deletion, copy number variation, inversion, or translocation.

102. The method of claim 100, wherein the structure-based mutation occurs on one or more chromosomes selected from the group consisting of Ch2, Ch4, Ch9, Ch12, Ch17, and Ch20.

103. The method of claim 100, wherein the structure-based mutation occurs on one or more chromosomes selected from the group consisting of Ch2p23, Ch4q24, Ch20q11, Ch17q23, Ch9p24, Ch17p23, Ch17q25, Ch2q33, and Ch12p12.

104. The method of claim 82, further comprising identifying in the pluripotent cells a structure-based mutation occurring on one or more chromosomes selected from the group consisting of Ch1, Ch12, Ch17q, CH20q11, and X-chromosome.

105. The method of claim 82, further comprising identifying in the received cells a structure-based mutation occurring on one or more chromosomes selected from the group consisting of Ch3, Ch4, Ch5, Ch7, Ch8, Ch9, Ch11, Ch12, Ch13, Ch14, and Ch18.

106. A method of evaluating quality of cells comprising:
a. receiving a sample of starter cells prior to a manufacturing process, wherein the starter cells are HSCs or T cells;
b. sequencing at least part of the genome of the starter cells; and
c. identifying in the starter cells a defect in one or more genes selected from the group consisting of DNMT3A, TET2, ASXL1, PPM1D, JAK2, TP53, SRSF2, KRAS and SF3B1.

107. The method of claim 106, wherein the starter cells are obtained from a blood sample.

108. The method of claim 106, wherein the starter cells are obtained from a subject.

109. The method of claim 106, wherein the starter cells are HSCs derived from a blood sample of a subject in need of treatment.

110. The method of claim 106, wherein the starter cells are HSCs derived from a blood sample of a donor subject.

111. The method of claim 106, wherein the starter cells are T cells derived from a blood sample of a subject in need of treatment.

112. The method of claim 106, wherein the starter cells are T cells derived from a blood sample of donor subject.

113. The method of claim 106, wherein the defect is a sequence-based mutation.

114. The method of claim 113, wherein the sequence-based mutation is a mis-sense mutation, silent mutation, frame-shift mutation, nonsense mutation, insertion mutation, deletion mutation, or splice-site disruption.

115. The method of claim 106, wherein the defect is in DNMT3A in exons 7 to 23.

116. The method of claim 106, wherein the defect is a mis-sense mutation in DNMT3A selected from the group consisting of G543C, S714C, F732C, Y735C, R736C, R749C, F751C, W753C, and L889C.

117. The method of claim 106, wherein the defect is a V617F mutation in JAK2.

118. The method of claim 106, wherein the defect is a disruptive mutation in TET2.

119. The method of claim 106, wherein the defect is a disruptive mutation in PPM1D.

120. The method of claim 106, wherein the defect is a mis-sense mutation in TP53 selected from the group consisting of R175H, G245S, R248W, R273G, P151S, R181H, H193R, M237I, G245C, R248Q, R267W, and R273L.

121. The method of claim 106, wherein the one or more genes are associated with cancer.

122. The method of claim 121, wherein the one or more genes are selected from the group consisting of DNMT3A, TET2, ASXL1, PPM1D, JAK2, SF3B1, SRSF2, and TP53.

123. The method of claim 121, wherein the one or more genes are selected from the group consisting of DNMT3A, TET2, and ASXL1.

124. The method of claim 106, further comprising identifying in the starter cells a sequence-based mutation in one or more genes selected from the group consisting of TERT and CHEK2.

125. The method of claim 106, further comprising identifying in the starter cells a defect in one or more genes selected from the group consisting of CBL, KMT2C, ATM, CHEK2, KDR, MGA, DNMT3B, ARID2, SH2B3, MPL, RAD21, SRSF2, and CCND2.

126. The method of claim 106, further comprising identifying in the starter cells a defect in one or more genes selected from the group consisting of HPRT, JAK1, JAK3, SLAMF6, IRF1, PLRG1, STAT3, and Notch1.

127. The method of claim 106, further comprising identifying in the starter cells a structure-based mutation in one or more genes selected from the group consisting of DNMT3A, TET2, ASXL1, PPM1D, JAK2, TP53, SRSF2, KRAS and SF3B1.

128. The method of claim 127, wherein the structure-based mutation is a duplication, deletion, copy number variation, inversion, or translocation.

129. The method of claim 127, wherein the structure-based mutation occurs on one or more chromosomes selected from the group consisting of Ch2, Ch4, Ch9, Ch12, Ch17, and Ch20.

130. The method of claim 127, wherein the structure-based mutation occurs on one or more chromosomes selected from the group consisting of Ch2p23, Ch4q24, Ch20q11, Ch17q23, Ch9p24, Ch17p23, Ch17q25, Ch2q33, and Ch12p12.

131. A method of evaluating quality of manufactured cells comprising:
a. receiving a sample of manufactured cells obtained upon completion of a manufacturing process;
b. sequencing at least part of the genome of the manufactured cells; and
c. identifying in the manufactured cells a defect in one or more genes selected from the group consisting of DNMT3A, TET2, ASXL1, PPM1D, JAK2, TP53, SRSF2, KRAS and SF3B1.

132. The method of claim 131, wherein the manufactured cells are manufactured from a population of pluripotent cells or somatic cells.

133. The method of claim 131, wherein the manufactured cells are manufactured from a population of hematopoietic stem cells (HSCs) or T cells.

134. The method of claim 131, wherein the defect is a sequence-based mutation.

135. The method of claim 134, wherein the sequence-based mutation is a mis-sense mutation, silent mutation, frame-shift mutation, nonsense mutation, insertion mutation, deletion mutation, or splice-site disruption.

136. The method of claim 131, wherein the defect is in DNMT3A in exons 7 to 23.

137. The method of claim 131, wherein the defect is a mis-sense mutation in DNMT3A selected from the group consisting of G543C, S714C, F732C, Y735C, R736C, R749C, F751C, W753C, and L889C.

138. The method of claim 131, wherein the defect is a V617F mutation in JAK2.

139. The method of claim 131, wherein the defect is a disruptive mutation in TET2.

140. The method of claim 131, wherein the defect is a disruptive mutation in PPM1D.

141. The method of claim 131, wherein the defect is a mis-sense mutation in TP53 selected from the group consisting of R175H, G245S, R248W, R273G, P151S, R181H, H193R, M237I, G245C, R248Q, R267W, and R273L.

142. The method of claim 131, wherein the one or more genes are associated with cancer.

143. The method of claim 142, wherein the one or more genes are selected from the group consisting of DNMT3A, TET2, ASXL1, PPM1D, JAK2, SF3B1, SRSF2, and TP53.

144. The method of claim 142, wherein the one or more genes are selected from the group consisting of DNMT3A, TET2, and ASXL1.

145. The method of claim 131, wherein the one or more genes are associated with tumorigenesis.

146. The method of claim 145, wherein the one or more genes are selected from the group consisting of TP53, KRAS, ASXL1, JAK2, SFSR2, and SFSB1.

147. The method of claim 145, wherein the one or more genes are selected from the group consisting of TP53 and KRAS.

148. The method of claim 131, wherein the one or more genes are associated with blood cancer.

149. The method of claim 148, wherein the one or more genes are selected from the group consisting of TET2 and DNMT3A.

150. The method of claim 131, further comprising identifying in the manufactured cells a defect in one or more genes selected from the group consisting of PCM1, HIF1A, and APC.

151. The method of claim 150, wherein the defect is a sequence-based mutation.

152. The method of claim 131, further comprising identifying in the manufactured cells a sequence-based mutation in one or more genes selected from the group consisting of TERT and CHEK2.

153. The method of claim 131, further comprising identifying in the manufactured cells a defect in one or more genes selected from the group consisting of CBL, KMT2C, ATM, CHEK2, KDR, MGA, DNMT3B, ARID2, SH2B3, MPL, RAD21, SRSF2, and CCND2.

154. The method of claim 131, further comprising identifying in the manufactured cells a defect in one or more genes selected from the group consisting of HPRT, JAK1, JAK3, SLAMF6, IRF1, PLRG1, STAT3, and Notch1.

155. The method of claim 131, further comprising identifying in the pluripotent cells a structure-based mutation in one or more genes selected from the group consisting of DNMT3A, TET2, ASXL1, PPM1D, JAK2, TP53, SRSF2, KRAS and SF3B1.

156. The method of claim 155, wherein the structure-based mutation is a duplication, deletion, copy number variation, inversion, or translocation.

157. The method of claim 155, wherein the structure-based mutation occurs on one or more chromosomes selected from the group consisting of Ch2, Ch4, Ch9, Ch12, Ch17, and Ch20.

158. The method of claim 155, wherein the structure-based mutation occurs on one or more chromosomes selected from the group consisting of Ch2p23, Ch4q24, Ch20q11, Ch17q23, Ch9p24, Ch17p23, Ch17q25, Ch2q33, and Ch12p12.

159. The method of claim 155, further comprising identifying in the pluripotent cells a structure-based mutation occurring on one or more chromosomes selected from the group consisting of Ch1, Ch12, Ch17q, CH20q11, and X-chromosome.

160. The method of claim 155, further comprising identifying in the received cells a structure-based mutation occurring on one or more chromosomes selected from the group consisting of Ch3, Ch4, Ch5, Ch7, Ch8, Ch9, Ch11, Ch12, Ch13, Ch14, and Ch18.

161. The method of claim 131, further comprising isolating a subpopulation of the manufactured cells that exhibit no identified defects in the one or more genes.

162. The method of claim 161, further comprising administering to a subject the isolated manufactured cells that exhibit no identified defects in the one or more genes.

163. The method of claim 162, wherein the isolated manufactured cells are administered to the subject to treat a disease or disorder.

164. The method of claim 163, wherein the disease or disorder is a blood, immune, metabolic, neurologic, or cardiovascular disorder.

165. The method of claim 163, wherein the disease or disorder is a cancer.

166. The method of claim 163, wherein the disease or disorder is selected from the group consisting of acute myeloid leukemia, acute lymphoblastic leukemia, myelodysplastic syndrome, myeloproliferative neoplasm, germ cell tumor, neuroblastoma, Ewing sarcoma, and medulloblastoma.

167. The method of claim 163, wherein the disease or disorder is a solid tumor.

168. The method of claim 163, wherein the solid tumor is a non-malignant tumor.

169. The method of claim 163, wherein the solid tumor is a malignant tumor.

170. The method of claim 131, further comprising isolating a subpopulation of manufactured cells that exhibit a defect in the one or more genes.

171. The method of claim 170, further comprising correcting the defect in the one or more genes.

172. The method of claim 171, further comprising administering to a subject the corrected isolated manufactured cells.
